(19) United States
(12) Patent Application Publication
(10) Pub. No.: US 2006/0275757 A1
(43) Pub. Date: Dec. 7, 2006

(54) Magnetic nanomaterials and methods for detection of biological materials

(76) Inventors: Gil U. Lee, West Lafayette, IN (US); Ronald P. Andres, West Lafayette, IN (US)

Correspondence Address: MUETING, RAASCH & GEBHARDT, P.A., P.O. BOX 581415, MINNEAPOLIS, MN, 55458, US

(21) Appl. No.: 10/519,021

(22) PCT Filed: Jun. 30, 2003
(86) PCT No.: PCT/US03/20226

§ 371 Date: May 3, 2006

Related U.S. Application Data
(60) Provisional application No. 60/392,192, filed on Jun. 28, 2002.

(30) Foreign Application Priority Data
Feb. 24, 2003 (US) 10/373609
Feb. 24, 2003 (US) 10/373600

(51) Int. Cl. C12Q 1/68; G01N 33/551; G01N 33/53

(52) U.S. Cl. 435/6; 435/7.1; 436/524; 977/900; 977/924

(57) ABSTRACT

Biological material in a sample is reacted with a novel functionalized superparamagnetic Fe/Au nanoparticle that specifically binds to the biological material in solution to produce a magnetic particle/biological material complex. The biological material is detected upon application of an external magnetic field which separates the magnetic bound complex from other components of the reaction mixture.

### RELATED APPLICATION

This application claims the benefit of U.S. Provisional Application Ser. No. 60/392,192, filed 28 Jun. 2002, U.S. patent Ser. No. 10/373,609 filed Feb. 24, 2003, entitled Fe/Au Nanoparticles and Methods and U.S. patent Ser. No. 10/373,600 also filed on Feb. 24, 2003 and entitled Magnetic Nanomaterials and Methods for Detection of Biological Materials, all of which are incorporated herein by reference in their entirety.

### BACKGROUND OF THE INVENTION

Current pathogen detection technologies are based on techniques that have been developed to support medical diagnoses. Traditionally, protein markers associated with pathogens have been identified using enzyme linked immunological solid-phase assays (ELISA). More recently, polymerase chain reaction coupled to fluorescence amplification have been used to identify genetic tags associated with a specific pathogen. The most advanced detectors based on these technologies can identify pathogenic agents at or below their lethal dose in less than 30 minutes. Unfortunately, these detectors are not widely available due to the cost of the instrumentation (fully automated instrumentation cost significantly more than $100,000) and operation (continuous use of an instrument can cost $10,000 per day and requires a trained technical staff). Further, many pathogens cannot be identified at lethal dose levels.

Magnetic materials are playing an increasingly important role in biotechnology due to the development of paramagnetic microparticles that are functionalized with specific binding moieties. Magnetic separation is known for the isolation of specific cell lines or polynucleotides from a growth medium or cell lysate using specific molecular receptors (i.e., binding agents) immobilized on magnetic carriers. This is done by adding a minute quantity of functionalized magnetic carrier to the target material (i.e., the growth medium or the cell lysate containing the analyte of interest) and using a strong magnet to immobilize the analyte-magnetic carrier complex on the wall of the separation vessel while the aqueous solution is removed. Cell separation using magnetic particles has proven to be a commercial success due to the high efficiency, high cell viability, and low cost of this process. Magnetic particles have also been used to detect pathogens in solid-phase assays based on force (D. R. Baselt et al., Vac. Sci. Technol., B14, 789-794, 1996), optical (G. U Lee et al., Bioanalytical Chemistry, 287, 261-271, 2000), and magnetic (D. R. Baselt et al., Biosensor Bioelectronics, 13, 731-739, 1998) amplification.

The magnetic carriers used in current bio-magnetic applications suffer from a number of deficiencies that limit their utility for the detection of biological materials, such as pathogen detection and identification. Because of their relatively low magnetic susceptibility on a volume basis, particles larger than the size of most pathogenic agents must be used in order to manipulate the pathogen/particle complex in a magnetic field. Prior art magnetic carriers consist of magnetic iron or iron oxide particles coated with or embedded in a polymer matrix and are typically micron sized. Magnetic particle/target complexes cannot easily be distinguished or separated from those magnetic particles not attached to a target species because of the large size of the magnetic particles. The prior art provides no way to determine whether the micron-scale magnetic particles that are collected have biological targets attached to them unless the biological target is large enough so that it is distinguishable from the magnetic particle(s) attached to it or there is a detection method that is specific for the target material. Small biological targets (e.g. DNA) may be amplified to facilitate detection, but this adds time and cost to the detection method. Thus, the prior art techniques are particularly problematic when applied to the detection of many important biological targets.

There is a need for functionalized, nanometer-size, magnetic carriers with large enough magnetic susceptibilities to permit manipulation of the pathogen/particle complex and an optical signature allowing for optical identification of single pathogen/particle complexes.

### SUMMARY OF THE INVENTION

The invention provides a highly sensitive and economical pathogen identification system using a new class of magnetic carriers to separate and detect pathogens. Functionalized nanoparticles that act as magnetic transducers are assembled from highly uniform nano-scale iron/gold (Fe/Au) particles functionalized with binding agents that bind the target biological material. The bound complex formed upon binding of the magnetic transducer to the target material is referred to herein as the “bound transducer complex”. Binding of the magnetic transducer to the biological target can be covalent or noncovalent (e.g., via ionic or hydrophobic interactions). The presence of the biological target is determined by optical detection of the bound transducer complex.

The Fe/Au nanoparticles that form the basis for constructing this new class of magnetic carriers can be synthesized with uniform particle diameters as small as a few nanometers. They are superparamgnetic at room temperature with a large magnetic susceptibility on a volume basis and this magnetic susceptibility can be controlled by varying the ratio of Fe to Au atoms in the particle. The Fe/Au nanoparticles have extremely small optical scattering cross sections. However, they can be optically detected by incorporating optically active species (e.g. optically active molecules, semiconductor nanoparticle quantum dots, or in a preferred embodiment Au nanoparticles) as part of the magnetic transducer particle.

Fe/Au and Au nanoparticles can be functionalized to selectively complex with specific biololgical targets and with each other. This capability is used in several immunoassay schemes in accordance with the present invention. The schemes include: i) direct binding assay where the target material or antigen binds to the magnetic transducer in solution, or ii) competitive binding assay wherein the target material or antigen competes for or displaces a labeled antigen or ligand on the magnetic transducer or a Au nanoparticle. In either of the assay schemes, the magnetic properties of the bound transducer complex can be used to separate the bound target molecule from the bulk sample. The separated bound transducer complex can be quantified by using detection methods that include: i) optically detecting the bound transducer complex ii) optically detecting a Au nanoparticle either as a free particle, a part of the bound transducer complex, or attached to the desired target material or a displaced ligand; iii) using the magnetic properties and/or change in the magnetic moment of the bound transducer complex compared to the free magnetic transducer particle to identify the bound transducer complex, and iv) using detection methods specific for the target material, which are common in the art.

The present invention includes a method of analyzing a sample for a target material using one or more the immunoassay techniques. The method comprises: preparing a magnetic transducer comprising a magnetic susceptible nanoparticle having at least one binding agent attached thereto wherein the binding agent is selected to bind to the target material in the sample. A labeled binding partner capable of binding to the binding agent is provided as well. The magnetic transducer and the labeled binding partner to the sample either separately or mixed together as a bound complex. The target material in the sample either competes with or displaces the labeled binding partner from binding to the binding agent on the nanoparticle.

In one embodiment, the present invention provides a method for analyzing a sample for a target material. The method comprises preparing a magnetic transducer that includes a magnetic susceptible nanoparticle which has at least one binding agent attached thereto. The binding agent can be said binding agent selected to bind to the target material in the sample;

providing a labeled binding partner capable of binding to the binding agent; and

adding the magnetic transducer and the labeled binding partner to the sample

Importantly, the bound transducer complex can be manipulated by an external magnetic field and can be separated from non-magnetic species (i.e., species that cannot be manipulated by an external field) and concentrated. Such non-magnetic species are also called diamagnetic species. The method of detection of the invention involves contacting a biological sample with a uniform population of magnetic transducers functionalized so as to bind a biological target, then applying a magnetic field to separate the bound transducer complex from other components of the sample. If the biological target is present in the sample, a bound transducer complex will form and will be mobile in the magnetic field. Advantageously, separation of the bound transducer complex from other sample components can be performed in an aqueous environment, thereby avoiding the use of a polymer matrix as in electrophoretic separations.

In one embodiment of the detection method, the sample is subjected to a magnetic field and the bound transducer complex is separated from the free (unbound) magnetic transducers. The bound transducer complex is differentiated from free (unbound) magnetic transducers based on its mobility in a known magnetic field. As the mobility of a magnetic particle in a liquid is a function of the magnetic force on the particle and the hydrodynamic radius of the particle, this embodiment assumes that the target species is large enough relative to the free magnetic transducer that it imparts a measurable increase in the particle's hydrodynamic radius and that the magnetic particles can be detected, either by reference to the relative mobility of a standard, by optical detection, or in some other way. Attachment of multiple magnetic transducers (polyvalent binding) to the biological target can cause greater relative differences in mobility.

Accordingly, the present invention includes a method for detecting a magnetic particle. The method comprises placing a first magnetic particle at a first location in a fluid medium; applying a magnetic flux through a portion of the medium including the first location; and observing movement of the magnetic particle in the fluid medium from the first location to a second location.

In another embodiment of the detection method, the biological target is contacted with two different populations of functionalized nanoparticles: a uniform population of functionalized magnetic transducer (Fe/Au) nanoparticles and a uniform population of functionalized optical transducer (Au) nanoparticles. The diamagnetic Au nanoparticles are functionalized so as to specifically bind with the biological target but not to complex with the Fe/Au nanoparticles (i.e. not to exhibit nonspecific binding). Application of an external magnetic field separates the Au nanoparticles that are attached to a bound transducer complex from the free Au nanoparticles. In this embodiment it is not necessary to separate the bound transducer complex from the free (unbound) magnetic transducers as the Au nanoparticles are easy to differentiate from Fe/Au nanoparticles based on their optical signatures and detecting the presence of a Au nanoparticle is tantamount to detecting the presence of a bound biological target.

The invention is not limited by the type of biological material detected (the “target material”) or the type of binding agent used to functionalize the transducer. The target material can be, for example, a biomolecule such as a polypeptide, a polynucleotide, carbohydrate, lipid, or other biological molecule; a complex of two or more biomolecules; or a higher order biomaterial such as an organelle, a membrane, a cell or a complex of cells. The binding agent can be an ion, a functional group or chemical moiety, or a larger molecular structure such as a functionalized polymer or a biomolecule such as a polypeptide, a polynucleotide, carbohydrate or a lipid. The defining characteristic of the binding agent is that it is capable of binding, with the desired level of specificity and selectivity, the intended target material.

The detection scheme that characterizes the invention is based on a simple homogeneous assay involving only solution phase reaction. It incorporates separation and concentration processes that make use of nanoscale magnetic transducers (i.e. functionalized Fe/Au nanoparticles) and optical detection involving nanoscale functionalized Fe/Au particles that have been made optically active or Au particles having strong optical signatures. The system is expected to achieve near single molecule sensitivity with minimal reagent consumption.

Accordingly, the invention provides a method for detecting a biological material in a sample that involves:

(a) contacting the biological material in the sample with a magnetic transducer comprising a single superparamagnetic Fe/Au nanoparticle comprising Fe atoms and Au atoms distributed in a solid solution with no observable segregation into Fe-rich or Au-rich phases or regions or a composite particle made up of such Fe/Au nanoparticles and an optically active species, and a binding agent that binds the biological material, to yield a reaction mixture comprising a bound transducer complex comprising the superparamagnetic nanoparticle and the biological material, and an unbound magnetic transducer;

(b) applying a magnetic field to separate the bound transducer complex from at least one other component of the reaction mixture; and

(c) detecting the bound transducer complex, wherein detection of the bound transducer complex is indicative of the presence of the biological material in the sample.

The magnetic transducer is characterized by a large magnetic susceptibility per particle volume, and can be synthesized with a uniform size and uniform magnetic and optical properties.

The magnetic transducer is functionalized with one or more binding agents, and the binding agents can be the same or different. Because of the possibility of multivalent functionalization, the bound transducer complex can include multiple magnetic transducers.

The invention further includes the magnetic transducers as described herein.

In one embodiment of the method for detecting a biological material, a bound transducer complex is detected by observing its relative magnetophoretic mobility in a magnetic field. The bound transducer complex can be separated from another magnetic component of the reaction mixture, including other bound transducer complexes and unbound magnetic transducers, or from other components, such as diamagnetic species. When multiple biological materials are to be detected, the sample is contacted with multiple magnetic transducers each functionalized to bind to a specific target.

The present invention also provides a method of analyzing a sample suspected of including a target material of interest. The method comprises: preparing a magnetic transducer comprising a Fe/Au nanoparticle functionalized with a first binding agent wherein the Fe/Au nanoparticle exhibits a first magnet moment; adding the magnetic transducer to the sample in an amount sufficient to bind to a target material in the sample and yield a bound transducer complex having the target material bonded thereto; and determining the magnetic moment exhibited by the Fe/Au nanoparticle of the bound transducer complex.

Additionally or alternatively, the bound transducer complex can be optically detected. If necessary, the superparamagnetic Fe/Au particles can be tagged with an optically active molecule, a semiconductor nanoparticle quantum dot or a Au nanoparticle to provide them with a resonant optical response. The bound transducer complex can be detected by optical tracking in a liquid or by collection on a substrate and imaging. Alternatively, the bound transducer complex can be collected on a substrate and detected using transmission electron microscopy.

In a preferred embodiment of the detection method of the invention, the biological material in the sample is also contacted with an optical marker functionalized with a binding agent that binds the biological material. The resulting reaction mixture includes a bound transducer complex that includes the magnetic transducer, the optical marker and the biological material; an unbound magnetic transducer; and an unbound optical marker. Application of a magnetic field causes the bound transducer complex to separate from the unbound optical marker. The binding agent of the functionalized optical marker binds only to the biological target, although in some applications it may be desirable for it to bind nonspecifically to the magnetic transducer. Detection of the optical marker in the bound transducer complex is indicative of the presence of the biological material in the sample. The binding agent of the magnetic transducer and the binding agent of the optical marker can be the same the same or different. In a particularly preferred embodiment, the functionalized optical marker is an Au particle functionalized with a binding agent that binds the biological target. Advantageously, use of an Au particle as an optical marker that binds the biological material allows detection of the bound transducer complex even in the presence of unbound magnetic transducers.

Accordingly, the present invention also includes a device for detecting the biological material. The device comprises a container that is configured to retain at least a portion of the sample wherein the container includes at least one wall having a magnet disposed adjacent thereto; and an optical detector that positioned next to the container to detect the present of one or more species in the sample.

Also included in the invention is a flow device for separating magnetic nanoparticles from diamagnetic nanoparticles. The device includes a channel comprising a recessed cavity comprising a substrate and a magnetic field adjacent the recessed cavity, and is operable to provide i) a liquid comprising magnetic and diamagnetic nanoparticles flowing through the cavity and ii) a diffusion barrier comprising a stagnant liquid layer in the recessed cavity, wherein the magnetic field provides for collection of magnetic nanoparticles on the substrate. The number of magnetic nanoparticles collected on the substrate is controlled by a process comprising controlling the flow rate of the liquid through the cavity. Additionally, the number of magnetic nanoparticles collected on the substrate is controlled by a process comprising controlling the thickness of the diffusion barrier, which in turn is controlled by controlling the depth of the recessed cavity. Also provided is a method for separating magnetic nanoparticles from diamagnetic nanoparticles that includes introducing a liquid comprising magnetic nanoparticles and diamagnetic nanoparticles in a channel comprising a recessed cavity comprising a substrate; selecting a flow rate of the liquid through the channel so as to create a diffusion barrier comprising a stagnant liquid layer in the recessed cavity; and applying a magnetic field adjacent the recessed cavity such that the magnetic nanoparticles are preferentially collected on the substrate.

The invention is further directed to a miniature instrument that separates and detects, and optionally identifies, biological materials, for example pathogens, in complex environmental matrices (such as air and water) with single molecule sensitivity. The mobility of the bound transducer complex in a magnetic field is used to separate it from the environmental matrix. The optical signature of the transducer complex is then used to detect the presence of a pathogen. For nucleic acid targets, nucleic fragments can be collected and detected in an optical microscope. For polypeptide targets, a miniaturized optical tracking system can be used to monitor separation and detection.

Accordingly, the invention further includes a device for detection of biological materials that includes means for magnetically separating components of a reaction mixture as described above, and means for detecting the bound transducer complex. In one embodiment, the detection means includes a means for detecting the optical signature of the bound transducer complex. In another embodiment, the detection means includes a means for detecting the relative magnetophoretic mobility of the bound transducer complex.

### BRIEF DESCRIPTION OF THE DRAWINGS

FIG. 1 is a schematic drawing of the arc region of a representative distributed arc cluster source (DACS) for use in synthesizing the Fe/Au nanoparticles.

FIG. 2 is a schematic drawing of a representative capture cell for use in capturing the Fe/Au nanoparticles as a stable suspension.

FIG. 3 is a schematic drawing indicating the random distribution of Fe atoms (dark spheres) and Au atoms (light spheres) on the surface of a 2.5 nm diameter Fe(10)/Au(90) nanoparticle.

FIG. 4 shows the atomic fraction of Fe in nanoparticles produced in the DACS as a function of the atomic fraction of Fe in the crucible.

FIG. 5 is a transmission electron microscope (TEM) micrograph of 10 nm diameter Fe(50)/Au(50) nanoparticles produced using the distributed arc cluster source of FIG. 1.

FIG. 6a is a graph showing a pair of magnetization curves (at 100K and 293K) of a bulk sample of Fe(50)/Au(50) particles over the range 0-60,000 Oe.

FIG. 6b is a graph showing a pair of magnetization curves (at 100K and 293K) of a bulk sample of Fe(50)/Au(50) particles over the range 0-1000 Oe.

FIG. 7 is a schematic drawing of Fe/Au nanoparticle surrounded by a protective monolayer of linear organic molecules, e.g., a mixed monolayer of dodecanethiol and dodecylamine, which provide colloidal stability in organic solvents.

FIG. 8 is a transmission electron microscope (TEM) micrograph of 150 nm diameter composite nanoparticles synthesized in solution from 10 nm Fe(50)/Au(50) particles and 4 nm Au nanoparticles.

FIG. 9 shows UV-Vis absorbance spectra for 20 nm diameter Au particles functionalized with DNA-B.

FIG. 10 shows UV-Vis absorbance spectra for 10 nm diameter Fe/Au particles taken both before and after functionalization with DNA-A.

FIG. 11 is a schematic drawing of (a) the experimental design to test whether Au and Fe/Au nanoparticles that have been functionalized with short, single-chain DNA sequences will hybridize with a complementary DNA molecule to form Fe/Au particle: DNA linker: Au particle complexes; and (b) the smallest Fe/Au particle:DNA linker:Au particle complex that forms during the experiments. Complexes made via DNA hybridization may contain multiple Fe/Au and/or Au nanoparticles do to multiple copies of the short DNA sequences attached to the nanoparticles.

FIG. 12 shows (a) a series of UV-Vis spectra showing the linking of 20 nm diameter Au nanoparticles functionalized with DNA-A and 20 nm diameter Au nanoparticles functionalized with DNA-B when the target DNA sequence is introduced into a buffered aqueous solution. a: the spectra for a stable colloidal mixture containing both of the functionalized Au particles before addition of the target DNA, b: the spectra for the colloidal mixture 240 minutes after addition of the DNA target, c: the spectra after heating the solution to cause reversible dehybridization of the Au particle: DNA linker: Au particle complexes; and (b) a series of UV-Vis spectra showing the linking of 10 nm diameter Fe/Au nanoparticles functionalized with DNA-A and 20 nm Au nanoparticles functionalized with DNA-B when the target DNA sequence is introduced into a buffered aqueous solution. a: the spectra taken when the DNA target is added to the mixture, b: the spectra taken 22 hours after addition of the target DNA, c: the spectra after heating the solution to cause reversible dehybridization of the Fe/Au particle: DNA linker: Au particle complexes.

FIG. 13 shows transmission electron microscopy images of (a) M13 phage; (b) M13 phage+anti-Mi3:Au particles; and (c) M13 phage+bovine serum albumin+anti-M13:Au particles.

FIG. 14 shows an experimental design for detecting M13 phage using anti-M13 conjugated Fe/Au particles and/or anti-M13 monoclonal conjugated Au particles.

FIG. 15 is a schematic drawing of one embodiment of magnetic capture cell.

FIG. 16 is a TEM micrograph showing a single 20 nm Au particle (a non-magnetic, i.e., diamagnetic, nanoparticle) captured from solution containing 2×10¹¹ (20 nm diameter) Au particles/mL and no Fe/Au (magnetic) nanoparticles.

FIG. 17 is a TEM micrograph showing a dense concentration of Fe/Au nanoparticles captured from a solution containing 2×10¹¹ (20 nm diameter) Au particles/mL and 2×10¹¹ Fe(50)/Au(50) particles/mL.

FIG. 18 is a TEM micrograph showing single Au particle detected in a large group of Fe/Au particles.

FIG. 19 is a diagrammatical illustration of another embodiment of a magnet capture cell for a fluid sample.

FIG. 20 is a diagrammatical illustration of one embodiment of a capture cell with a detector for analyzing Au nanoparticles in suspended solution.

### DETAILED DESCRIPTION

We have developed a unique synthesis procedure capable of producing magnetic nanoparticles having a controlled size and shape; having a large and stable magnetic moment; that do not corrode in high ionic strength aqueous solutions; and that allow chemical attachment of DNA, peptides, and other bio-molecules to their surface. These particles are size-selected spherical metal clusters of iron and gold (Fe/Au) with controlled diameters in the range of 10-50 nm and with Fe atomic fractions in the range of 0.0-0.70.

The invention has numerous advantages over the prior art. The functionalized magnetic transducers of the present invention are much smaller (nanoscale, for example between 10 and 100 nm, typically about 20 nm in diameter). These nanoparticles are superparamagnetic at room temperature with saturation magnetic moments and magnetic susceptibilities per volume that are much greater than prior art magnetic particles. In addition their magnetic characteristics can be modified by modifying the Fe:Au atomic ratio of the particles. Although as synthesized the Fe/Au particles have a relatively wide size distribution, functionalized Fe/Au particles can be size selected in solution to produce a population of nearly monodispersed nanoparticles. Fe/Au nanoparticles are resistant to oxidation in an aqueous environment.

The Fe/Au nanoparticle is superparamagnetic and may, in some embodiments, have one or more advantages the following advantages, including but not limited to:
1) The particles have a high degree of magnetization and a large magnetic susceptibility.
2) Because the surface of the particle contains a high density of gold atoms, a wide variety of organic molecules can be attached to the surface to impart colloidal stability or to link the particles to each other through the use of the well studied binding reaction of thiols and disulfides to gold surfaces. The particles can also be functionalized with a wide variety of biological moieties.
3) The presence of the Au atoms also protects the Fe atoms in the interior of the particle from oxidation.
4) The particle exhibits a uniform volume magnetization and, because the particle does not contain layers, shells or regions having different compositions, it can be synthesized as a truly nanoscale particle, i.e. a particle whose diameter is only a few nanometers. These particles are so small that they can function as “magnetic molecules” in certain biological applications.
5) The particles are superparamagnetic at room temperature, i.e. the unpaired electron spins due to the Fe atoms in the particle are coupled together to produce a net magnetic moment. The orientation of this magnetic moment is random in the absence of an external magnetic field. In the presence of an external magnetic field the magnetic moment aligns with the field
6) The magnetic moment of the Fe/Au particles is proportional to the number of Fe atoms in the particle. It can be varied independent of the particle diameter by varying the ratio of Fe to Au. For example at 293K, for 10 nm diameter Fe(50)/Au(50) nanoparticles the net saturation magnetic moment is ˜1 Bohr magneton per Fe atom in the particle or 22.5 emu/g.
7) The particles can be synthesized with a uniform particle diameter and a uniform atomic composition. The particle diameter can be accurately controlled in the range of about 5 nm to about 50 nm. The Fe atom concentration can be accurately controlled in the range of about 5 atom % to about 50 atom % (i.e., a range of about Fe(5)/Au(95) to about Fe(50)/Au(50)
8) The particles are stable against oxidation and can be functionalized so that they are soluble in either organic solvents (i.e. they can be made hydrophobic) or water (i.e. they can be made hydrophilic).
9) The nanoparticles are expected to be nontoxic. The nanoparticle or nanoparticle core consists of only Fe atoms and Au atoms which are generally considered to be biocompatible. In addition, the immunogenicity of the Fe/Au nanoparticles is expected to be low. Since many immunological responses rely on surface antigen recognition, the small size and surface area of the Fe/Au nanoparticles are expected to limit non-specific protein binding and hence the host's immunological response.
10) The Fe/Au nanoparticles have small optical scattering cross sections and this property is advantageous in some bio-separation applications.
   Fe/Au Nanoparticle Production

The Fe/Au nanoparticles are produced in a distributed arc cluster source (DACS). This is an updated version of the aerosol reactor first proposed by Mahoney and Andres (Materials Science and Engineering A204, 160-164 (1995)). This new apparatus is designed to produce colloidal suspensions of metal nanoparticles having diameters in the 5-50 nm size range. The DACS is a gas aggregation reactor, which employs forced convective flow of an inert gas to control particle nucleation and growth. It is capable of producing equiaxed nanoparticles of almost any metal or metal mixture with a fairly narrow size distribution and is capable of achieving gram per hour production rates.

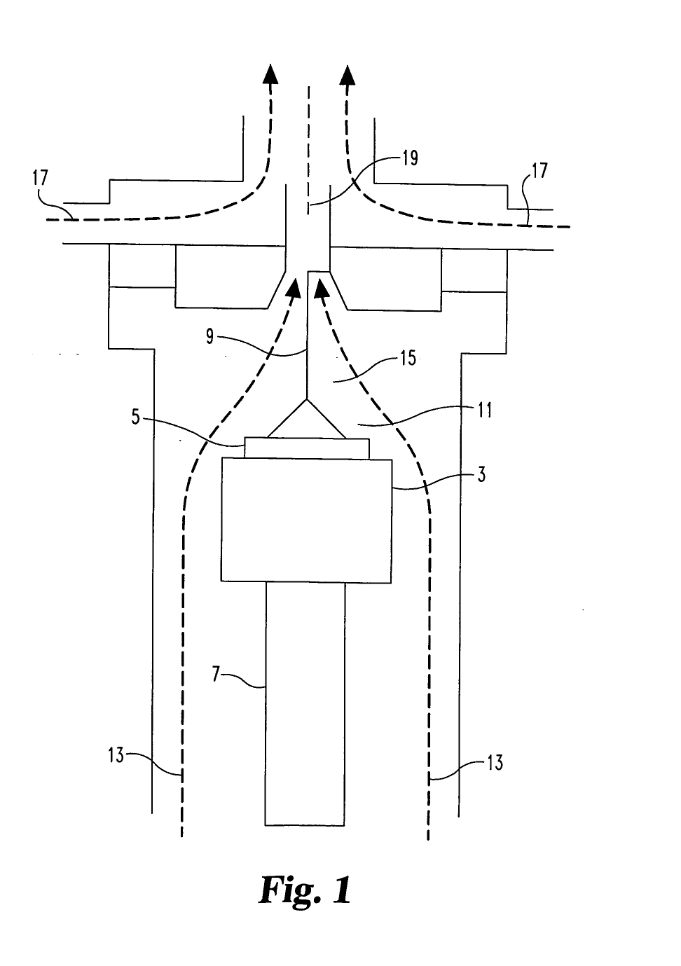

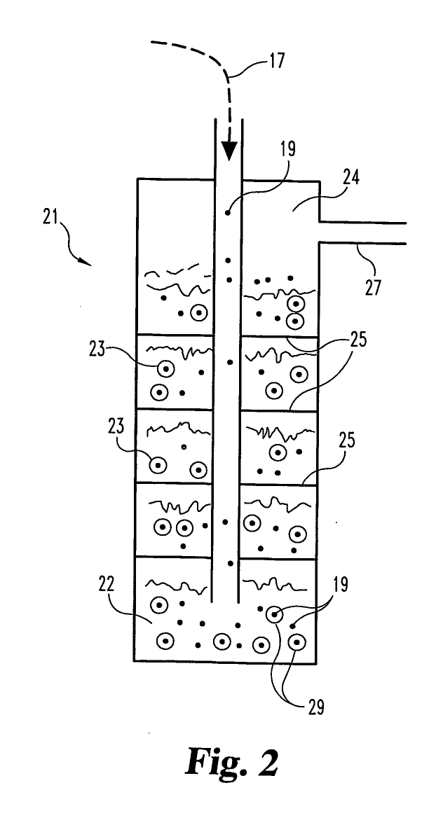

FIG. 1 shows a distributed arc cluster source 1 for use in synthesizing the superparamagnetic Fe/Au nanoparticles. Tungsten feed crucible 3 is surrounded by tantalum shield 5 and mounted on positive biased carbon rod 7 in proximity to tungsten electrode 9. A metal or metal mixture is placed in open tungsten crucible 3, and this metal charge is evaporated by means of an atmospheric pressure direct current (d.c.) arc discharge 11 established between the melt and the tungsten electrode 9. Carrier gas flow 13, room temperature argon, entrains the evaporating metal atoms 15 and rapidly cools and dilutes the metal vapor, causing solid particles to nucleate and grow. Particles 19 are produced as bare metal clusters entrained in the gas; the synthetic process leaves their surfaces free of organic molecules of any kind and ready for functionalization. Quench gas flow 17, room temperature helium or nitrogen, further cools the particles 19 and transports them to a vessel where they are contacted with a liquid and captured as a colloidal suspension (see FIG. 2) rather than being deposited on a substrate.

The mean size of the particles is a function of both the metal evaporation rate, which is controlled by the power to the arc, and the gas flow rates. Preferably, the nanoparticles have a mean diameter of about 5 nm to about 50nm and a variance of less than 50% of the mean. Size-selective precipitation can be used to reduce the variance, e.g., to approximately 5% of the mean.

The mean composition of the particles (in the case of a mixed metal charge) depends on the relative evaporation rates of the components in the charge and is a function of the composition of the molten mixture in the crucible. In the present case this is a mixture of Fe and Au of known composition. A specific composition in the crucible yields a specific particle composition.

FIG. 2 shows an embodiment of capture cell 21 used in the synthesis of the nanoparticles according to the invention. Capture cell 21 contains multiple liquid-filled vertical chambers 23 connected by baffle plates 25. Quench gas 17 carrying nanoparticles 19 from the distributed arc cluster source (FIG. 1) is injected into the liquid contained in capture cell 21. Nanoparticles 19 are captured in liquid in the bottom chamber 22 and percolate up through the liquid in successive vertical chambers 23. Gas bubbles rise 29 and contact baffle plates 25 as they enter the vertically adjacent chamber 23. As they rise in a liquid gas bubbles naturally coalesce, and gas may build up underneath the baffle plate 25. Perforated baffles break up the gas into smaller bubbles each time it passes through a baffle plate. This gives the particles 19 still entrained in the gas more opportunity to contact, and thereby transfer into, the liquid. Quench gas 17 exhausts from outlet 27 in the uppermost vertical chamber 24. The liquid in capture cell 21 is well mixed by the gas flow and there is no segregation of particles 19 in the different chambers as defined by baffle plates 25 is typically observed.

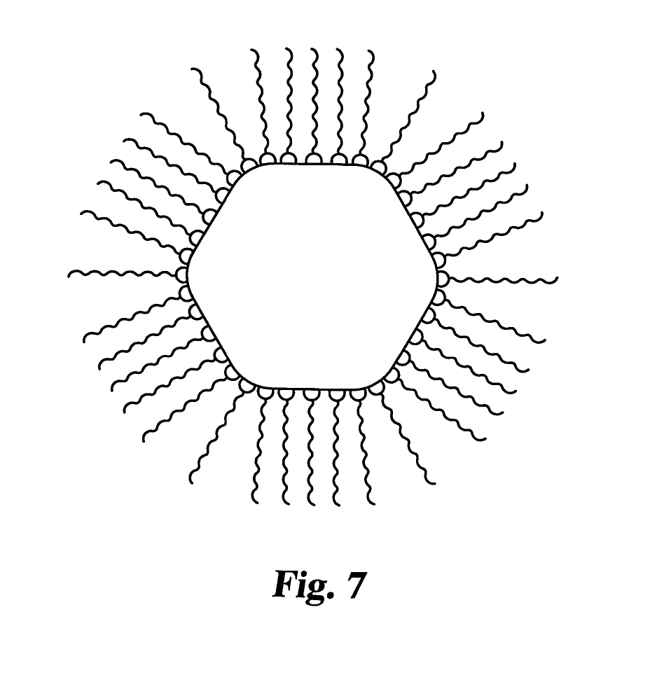

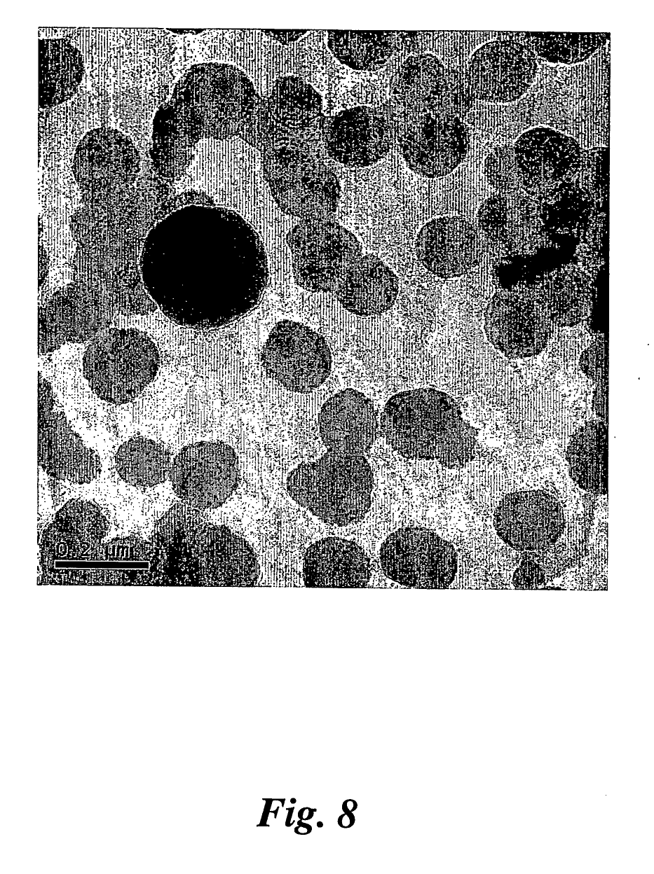

In one embodiment, nanoparticles 19 are captured in an organic solvent. When capturing particles in an organic solvent such as mesitylene, additional molecules that rapidly coat the particles with a covalently attached monolayer, such as dodecanethiol and dodecylamine (FIG. 7) are preferably added to the solvent, for example at a concentration of about 1.0 mM). These organic additives attach directly to particles 19 and protect them from aggregating in capture cell 21.

In another embodiment, nanoparticles 19 are captured in an aqueous liquid such as a dilute sodium citrate solution to produce a charge-stabilized nanocolloid. The negative citrate ions form a diffuse layer around the metal nanoparticles and keep them in suspension without aggregation. This is also a convenient starting point for further functionalization reactions. Optionally, one or more organic molecules such dodecanethiol and dodecylamine can be added, typically with a cosolvent, such as ethanol, to the citrate stabilized suspension. When these organic molecules react with the Fe/Au particles in an aqueous environment, they cause the particles to flocculate and drop out of solution. The particles can then be dried and re-suspended in an organic solvent such as dichloromethane, and have been shown to be equivalent to particles captured directly in an organic solvent in which dodecanethiol and dodecylamine have been added.

In yet another embodiment, nanoparticles 19 are captured in an aqueous liquid such as a dilute sodium citrate solution that contains one or more functionalizing molecules, allowing capture of the charge-stabilized nanocolloid and functionalization to be performed in a single step rather than in successive steps.

The resulting colloidal suspensions are stable for weeks and the particles can be stored in this state.

Diameter and Composition of the Fe/Au Nanoparicles

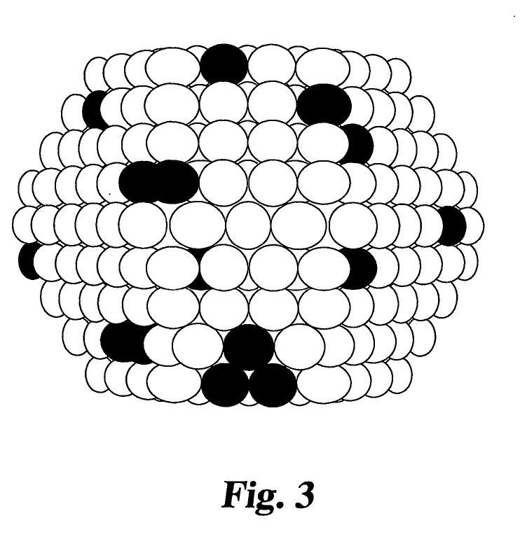

The Fe/Au nanoparticles are defined herein by the diameter and composition of the Fe/Au nanoparticle or, in the case of a functionalized nanoparticle, the Fe/Au metal core. For example, “10 nm diameter Fe(60)/Au(40)” indicates a particle (or metal core) with a 10 nm diameter core having an atomic composition 60% Fe atoms and 40% Au atoms. In a preferred embodiment, the Fe atoms and the Au atoms are distributed randomly within the nanoparticle or nanoparticle core (FIG. 3).

Diameters of the Fe/Au nanoparticles are preferably at least about 5 nm and at most about 50 nm, although particles smaller (e.g., diameter of about 2.5 nm) or larger (e.g., diameter of about 100 nm) can be produced using the method described herein.

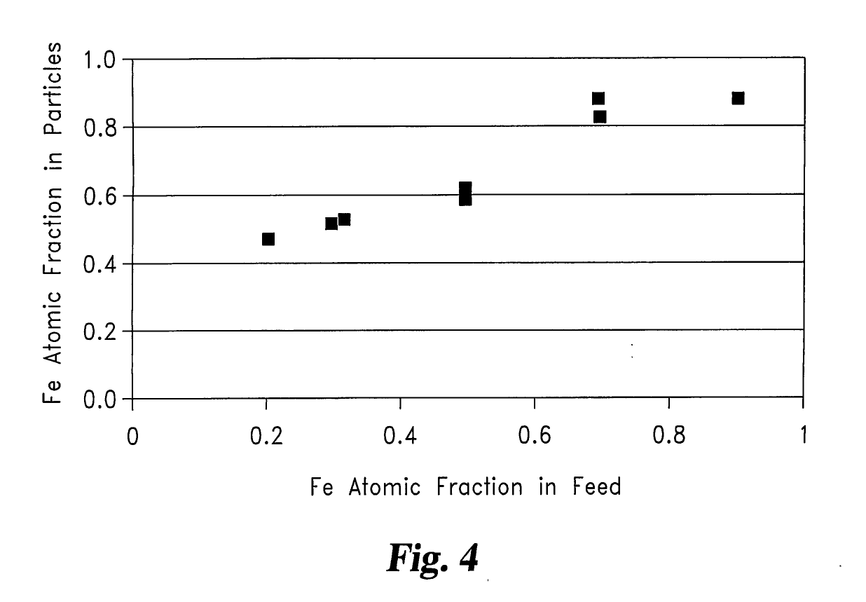

Atomic adsorption experiments made by dissolving a large number of identical Fe/Au particles in acid can used to determine their composition. Transmission electron microscope images made by supporting large numbers of the same Fe/Au particles on thin carbon membranes have shown that the particles have an essentially random distribution of Fe and Au atoms (i.e., the Fe and Au atoms do not segregate into observable Fe rich and Au rich regions or phases) as long as the Fe atom/Au atom ratio does not exceed about 7:3, i.e., Fe(70)/Au(30). Above 70 atomic % Fe however, phase segregation is observed. Particles with Fe atomic fractions of 50% or less were found to have reproducible magnetic characteristics and surface functionalization. FIG. 4 shows the Fe content of the particles as a function of the ratio of Fe to Au in the metal charge (feed).

The Fe content of the Fe/Au nanoparticles is preferably at least about 0.01%; (i.e. Fe(0.01)/Au(99.99)); more preferably it is at least about 5% (i.e., Fe(5)/Au(95)). At most, the Fe content of the nanoparticles is preferably about 70 atom % (i.e., Fe(70)/Au(30)); more preferably it is at most about 50% (i.e., Fe(50)/Au(50).

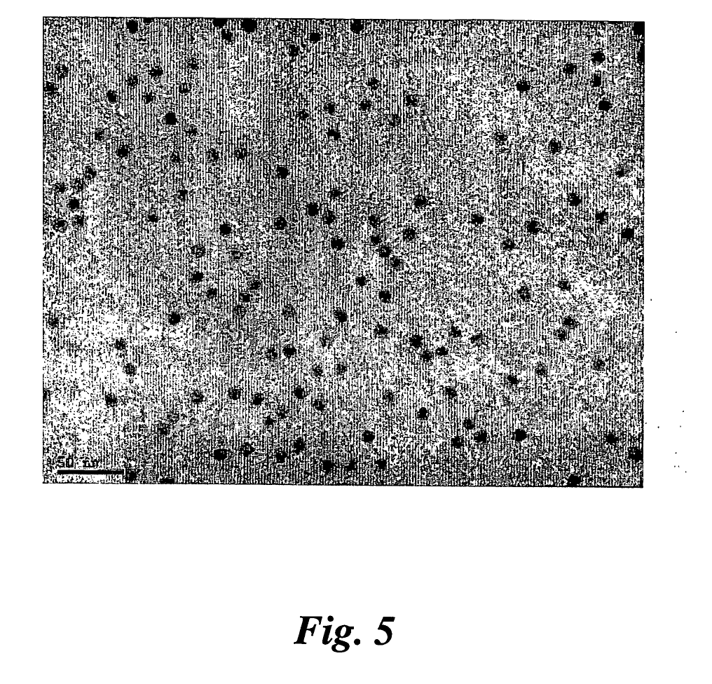

FIG. 5 shows a TEM micrograph of a sample of uniform 10 nm diameter Fe(50)/Au(50) particles. The nanoparticles were captured as a stable colloid by bubbling the aerosol stream from the DACS into distilled water containing sodium citrate. The particles were then coated with a mixed monolayer of dodecanethiol and dodecylamine molecules by adding dodecanethiol and dodecylamine in ethanol to the colloidal solution. The coated particles precipitated spontaneously from the aqueous solution, were dried and re-suspended in dichloromethane. The careful addition of acetonitrile, which is a poor solvent for the particles, was used to narrow the particle size distribution by size-selective precipitation. The TEM sample was obtained by spreading a drop of the dichloromethane solution on a copper TEM grid coated with a thin carbon film.

The nanoparticle is thought to contain only zero valent iron and gold, however, some of the Fe atoms, especially those on or near the surface, may be oxidized.

Magnetization

The relationship between the field experienced within a sample and the applied field is known as the magnetic susceptibility. Magnetic susceptibility is calculated as the ratio of the internal field to the applied field and represents the slope of the curve of magnetization (M) vs. magnetic field strength (H). It is typically expressed as volume susceptibility (emu/Oe-cm³, or simply, emu/cm³), mass susceptibility (emu/Oe-g, or emu/g) or molar susceptibility (emu/Oe-mol, or emu/mol).

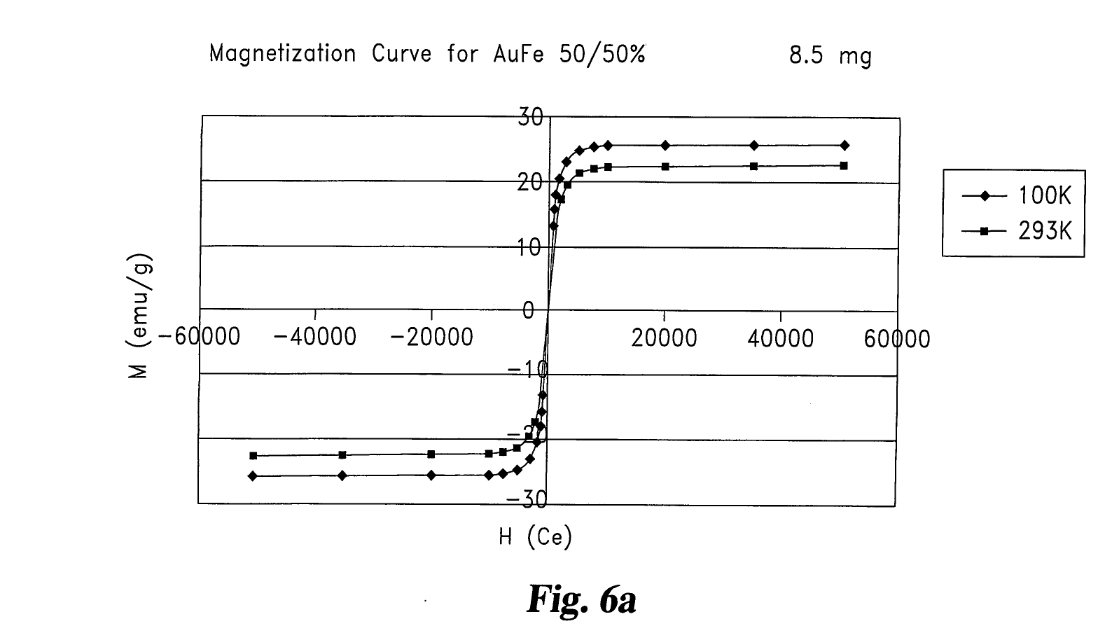

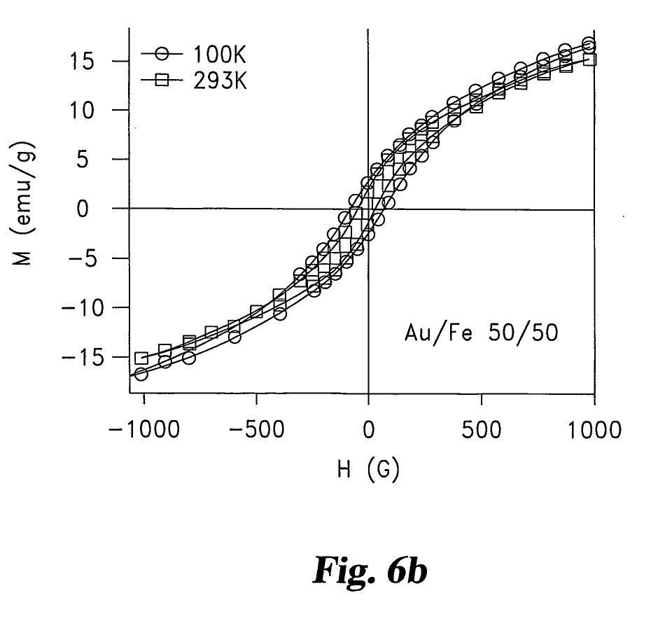

The nanoparticles exhibit strong magnetic susceptibility and stable magnetic characteristics. The magnetic characteristics of Fe/Au particles can be measured by capturing a sample of particles of known weight and measuring the magnetization curve of the bulk sample. The results of a representative experiment for the particles shown in FIG. 5 are shown in FIGS. 6a and 6b. Fe/Au particles with an average diameter of 10 nm and an Fe composition of 50 atom % (Fe(50)/Au(50)) were coated with a mixed monolayer of dodecanethiol and dodecylamine molecules and were magnetically collected from a mesitylene solution. They exhibited a saturation magnetization (attained when all magnetic moments in the sample are aligned) at 293K of 22.5 emu/g or 280 emu/cc (FIG. 6a). This is equivalent to a saturation magnetization of ˜100 emu/g Fe. The magnetic susceptibility of these nanoparticles is 0.2 emu/Oe-cm³ (emu/cm³) over the range 0-1,000 Oe and 0.25 emu/Oe-cm³ (emu/cm³) over the range 0-500 Oe (FIG. 6b). Prior art micron-scale nanoparticles have magnetic susceptibilities that are orders of magnitude less than the 0.1 to 0.2 emu/cm³ at 293K that characterizes the Fe/Au nanoparticles. Furthermore, the Fe/Au nanoparticles are not susceptible to loss of their magnetic properties due to the chemical transformation of magnetic iron oxides to diamagnetic Fe₂O₃ as are the prior art particles.

In addition, because the diameter and Fe/Au ratio of the particles can be accurately controlled, the magnetic moments of the Fe/Au particles can also be controlled. Magnetization curves similar to those shown in FIGS. 6a and 6b have been determined for samples of Fe/Au particles having different mean diameters and different compositions. These curves indicate that the Fe/Au nanoparticles are superparamagnetic with a saturation magnetic moment that, for a given mean diameter, is proportional to the Fe/Au ratio.

Surface Monolayers

The Fe/Au nanoparticles are initially produced as bare Fe/Au particles in a gas mixture of argon and nitrogen. It is frequently desirable to coat the particles with a monolayer of organic molecules to prevent nonspecific particle aggregation and/or to provide the functionality needed for an intended application. A wide range of organic molecules will react with the atoms on the surface of the Fe/Au particles to form a protective monolayer over the Fe/Au metal core. The preferred coating method depends on the structure of the organic molecule, its hydrophobic or hydrophilic nature, and whether the particles are captured in an aqueous or an organic solution. In a preferred embodiment, this is accomplished using thiol-terminated organic molecules so as to take advantage of the well-established reaction between thiol (—SH) and gold (Au).

When the organic molecules impart a hydrophilic nature to the surface of the particles, the particles are preferably first captured in a dilute aqueous solution of sodium citrate. This produces a charge-stabilized colloidal suspension that remains stable for many weeks. The organic molecules are subsequently added as a dilute solution to this colloidal suspension of charge-stabilized particles.

The attachment of organic molecules that impart a hydrophobic nature to the surface of the particles is preferably performed in either of two ways. When the organic ligand is water soluble or can be made soluble by the addition of a cosolvent such as ethanol, the particles are first captured in a dilute aqueous solution of sodium citrate as they are prior to functionalization with a hydrophilic ligand. The organic ligand is subsequently added to this colloidal suspension, optionally in the presence of a cosolvent, to react with the particles. Adding a linear alkanethiol to the liquid, for example, and a cosolvent such as ethanol (to increase the solubility of a hydrophobic ligand such as an alkanethiol), causes the particles to be rapidly coated with a monolayer of the alkanethiol. The thiol groups react with gold atoms on the surface of the Fe/Au particles and encapsulate the particles with a hydrophobic monolayer. The elimination of charge on the particles and the encapsulation of the particles by a hydrophobic monolayer causes the nanoparticles to aggregate and settle out of solution.

Once the coated particles are washed and air-dried, they can be re-suspended in an organic solvent such as dichloromethane or mesitylene (1,3,5-trimethyl-benzene). When re-suspended in an organic solvent the particles can be manipulated as stable physical entities and/or the alkanethiol molecules can be displaced by other organic thiols or dithiols. The Fe/Au particles encapsulated by a hydrophobic monolayer such as provided by a linear alkanethiol can be self-assembled into ordered arrays and molecularly linked together by bifunctional molecules such as conjugated dithiols or di-isonitriles to form thin films and bulk materials with interesting electrical and magnetic properties (Andres et al., Science 273, 1690 (1996)).

The second way in which organic molecules that impart a hydrophobic nature to the surface of the particles can be attached to the bare particles is to capture the particles directly in an organic solvent such as mesitylene in which one or more hydrophobic molecules such as dodecanethiol and/or dodecylamine have been added (Andres et al., Science 273, 1690 (1996)). Because of the presence of Fe atoms as well as Au atoms on the surface of Fe/Au particles, it is found that a mixed monolayer such as is produced by including both a thiol such as dodecanethiol and an amine such as dodecylamine provides the best encapsulation. When the particles are coated with an alkanethiol or other hydrophobic organic ligand monolayer and are suspended in an organic solvent, it is possible to cause them to aggregate and precipitate by adding a poor solvent such as ethanol or acetonitrile to the solution. Once the particles are air-dried they can be re-suspended in clean solvent and manipulated as described in the previous paragraph.

In addition to the functionalization with alkanethiols, other functionalization reactions that can conveniently be performed on charge-stabilized nanoparticles include, but are not limited to, adding a thiol-terminated polyethylene glycol (PEG) molecule to coat the particle with a hydrophilic monolayer, adding a DNA oligomer that is terminated by an linear alkane spacer and a thiol ligand, adding thiolpyridine to functionalize the particles with pyridine, and adding bis(p-sulfonatophenyl)phenyl phosphine for producing uniformly charged particles that are ideal for size selective separation of the particles in aqueous solution.

Notwithstanding the above, it should be understood that the invention is not limited by the type of linkage between the organic molecule and the metal core. For example, the linkage can be chemical or enzymatic, and can be covalent, ionic, or hydrophobic in nature.

For many applications, especially biological and biomedical applications, it is preferable to produce Fe/Au nanoparticles that are water-soluble. That is, they can be functionalized so that they remain hydrophilic. For example, it may be desirable to functionalize the Fe/Au core with DNA. This can be been done by adding to the citrate solution DNA oligomers that are terminated by a linear methylene sequence, a disulfide group, a second linear methylene sequence and an OH group (Nature 382, 607 (1996)). These DNA oligomers encapsulate the Fe/Au particles and produce stable physical entities that can be precipitated from the aqueous solution by adding excess electrolyte. Decanting the liquid and adding fresh water re-suspends the particles. Functionalizing the particles in this way with single-stranded DNA provides a method by which the Fe/Au particles can be selectively linked to each other, to other DNA functionalized particles, or to solid surfaces to produce composite structures with interesting properties.

Other hydrophilic molecules besides DNA can be attached to the particles by means of thiol or disulfide groups. For example a polyethylene glycol (PEG) polymer terminated by linear methylene sequence terminated a thiol group can be added to the citrate solution to form a hydrophilic coating on the particles, pyridinethiol can be added to the citrate solution to coat the particles with pyridine ligands, and a great variety of biomolecules such as proteins, nucleic acids, carbohydrates, lipids, etc. can be similarly attached to the particles. Higher order biomaterials such an organelles, a membranes, cells or a complexes of cells can also be bound to the Fe/Au particles.

Fe/Au nanoparticles functionalized with specific biological binding moieties are expected to have many in vitro applications such as separation and detection of biomaterials. Because these nanoparticles are expected to be nontoxic and can move freely in the human circulatory system they also are expected to have multiple in vivo biomedical diagnostic and therapeutic applications.

Although the surface of the Fe/Au nanoparticles contains Fe atoms as well as Au atoms, many of the protocols developed to functionalize Au nanoparticles with specific biomolecules and bioreceptors may be used with the Fe/Au nanoparticles to produce functionalized Fe/Au nanoparticles that are water-soluble. Most of these protocols start with bare Au nanoparticles in a dilute aqueous sodium citrate solution, and they are equally applicable to bare Fe/Au nanoparticles. As an example of this approach, the protocol developed by Mirkin and his co-workers (Nature 382, 607 (1996)) which has been used by us to successfully functionalize Fe/Au nanoparticles with DNA oligomers.

The binding of biomaterials to the Fe/Au particles can also be accomplished by ionic forces using for example thiol-alkyl-sulfate or thiol-alkyl-amine molecules to impart a negative or positive charge on the particles or by specific antigen/antibody binding.

The ability to precipitate and then re-suspend particles protected by a tightly bound organic monolayer provides a way to narrow the particle size distribution by means of size-selective precipitation. For example, when the Fe/Au particles are coated with a monolayer of DNA oligomers, the first particles to precipitate as the electrolyte concentration is increased are the largest particles. Similarly, for particles coated with a monolayer of linear alkanethiol molecules, the first particles to precipitate as a poor organic solvent is added are the largest particles. For example, subjecting a population of nanoparticles having a mean diameter of about 5 mm to about 50 nm to size-selective precipitation can decrease the variance from about 50% of the mean to approximately 5% of the mean, significantly narrowing the size distribution of the particle population.

Specific Binding of Magnetic Transducers to Target Materials

The Fe/Au nanoparticles described herein are uniquely suited for use in a wide variety of applications including biomagnetic and environmental magnetic applications. They can be produced in gram amounts as size selected spherical nanoparticles. Their magnetic moment, which can be controlled independently of size, is stable and large. The bare metal clusters can be converted into molecular protected particles that do not coagulate in high ionic strength aqueous solutions and various interesting molecules can be readily attached to the surface of the clusters via thiol linkers. Consequently, the Fe/Au nanoparticles can be tailored to bind to a wide variety of target materials.

The target materials can include any species of interest. Non limiting examples of target materials that can be detected and analyzed in accordance with the present invention include: proteins, peptides, carbohydrates polysaccharides, glycoproteins, lipids, hormones, receptors, antigens, allergens, antibodies, substrates, metabolites, cofactors, inhibitors, drugs, pharmaceuticals, nutrients, toxins, poisons, explosives, pesticides, chemical warfare agents, biohazardous agents, vitamins, heterocyclic aromatic compounds, carcinogens, mutagens, narcotics, amphetamines, barbiturates, hallucinogens, waste products, contaminants or other molecules. Molecules of any size can serve as targets. An analyte is not limited to a single molecule, but may also comprise complex aggregates of molecules, such as a virus, bacterium, spore, mold, yeast, algae, amoebae, dinoflagellate, unicellular organism, pathogen or cell.

The nanoparticles of the present invention find particularly useful application to bind to and detect biological targets.

In one embodiment, the magnetic transducer contains a nucleic acid binding agent, such as an oligonucleotide, and the target molecule is a nucleic acid such as DNA or RNA. Preferably the binding agent is a thiolated nucleic acid (typically a 3′ or 5′ thiolated nucleic acid), and the thiolated nucleic acid reacts with the Au atoms on the surface of the Fe/Au nanoparticles to form the magnetic transducer. For example, the Fe/Au nanoparticle can be functionalized with DNA and, optionally, one or more passivating monolayers to prevent nonspecific absorption, thereby producing magnetic transducers that complex with specific DNA sequences.

In another embodiment, the functionalized transducer is a magnetically labeled binding agent that binds a polypeptide. Such agents can be selected to bind a polypeptide (e.g., a peptide, an oligopeptide, or a protein or proteinaceous material) of any size and/or composition. The binding agents can be used to control the assembly of the magnetic clusters with nanometer precision in order to identify, for example, toxin and viral targets. Preferably, the binding agent is a peptide or an antibody. Both free peptide labeled nanoparticles as well as peptide labeled nanoparticles assembled on polysaccharide superstructures can act as magnetic transduction complexes for the identification of various biological materials such as toxin and viral targets. The structure of the polysaccharide transducers is based on the assembly of optically active dyes in amylose (L. S. Choi et al., Macromolecules, 31(26):9406-9408 (1998), but the dyes are replaced with magnetic clusters of defined size and magnetization.

Advantageously, a two stage chemistry can be used to functionalize the Fe/Au nanoparticles for interaction with polypeptides and other biomolecules. First, functional groups are incorporated on the surface to solubilize the nanoparticle, such as derivatization with alkanethiols having a T-terminal moiety that is highly polar, ionic, or strongly hydrophilic, such as an amine or a carbohydrate moiety. Such functional groups can be synthesized by reacting bromoalkanethiol with a trialkaylamine or the hydroxy-terminal of the saccharide under basic conditions, respectively. The choice of functional group influences the specific and nonspecific binding at the particle interface.

Functionalization of the particles with an agent that binds protein or DNA can be facilitated by adding a limited number of functional surface groups of a second kind. The existing alkanethiol can be replaced with a N-hydroxy-succinimide (NHS) alkanethiol, which has a chemistry designed to react with the primary amines of proteins and DNA molecules. Second, a portion of the functional groups can be modified or replaced with functional groups that specifically bind the target biomolecule.

Competitive Binding Assay

A competitive binding assay can be used to detect a target material in the sample. In this embodiment of the present invention, the Fe/Au nanoparticle can be functionalized with a binding agent selected to bind to the target material or antigen. A separate Au nanoparticle can be functionalized with the target material or a derivative thereof that is capable of binding to the binding agent.

A bound transducer complex comprising the functionalized Fe/Au nanoparticle and the functionalized Au nanoparticle are formed. Typically this will involve a covalent bond or electrostatic interaction between the binding agent of the Fe/Au nanoparticle and the target material (or derivative thereof) of the Au nanoparticle. This bound transducer complex can be added to the sample. The target material in the sample can displace the bound Au nanoparticle from the binding agent thereby releasing the Au nanoparticle into the bulk sample.

The Au nanoparticle in the sample can be detected optically, i.e, its optical signature can be detected in the bulk sample. This indicates that the target material is present in the sample and quantification of the optical signal can be used to determine the concentration of the target material when present.

Mobility of the Bound Transducer Complex in a Magnetic Field

The bound transducer complex of the invention can be manipulated in a magnetic field. The magnetic force experienced by a bound transducer complex in a magnetic field depends on the number of magnetic nanoparticles attached to the biological target and the magnetic susceptibilities of these nanoparticles. The mobility of this complex in an applied magnetic field is a function of 1) the total volume of the magnetic nanoparticles that are part of the complex and their Fe/Au ratios and 2) the hydrodynamic cross-section of the complex.

As noted above, magnetic separation is known for the isolation of specific cell lines or polynucleotides from a growth medium or cell lysate using specific molecular receptors (i.e., binding agents) immobilized on magnetic carriers. This is a rapid and highly economical process, but is limited in that only gross separations can be achieved.

One aspect of the invention derives from an observation that the mobility of magnetic carriers in a medium, such as, an aqueous solution, (“magnetophoresis”) can be used to identify a specific analyte much as mass is used to identify a specific analyte in mass spectrometry or as gel electrophoresis is used to separate and identify DNA fragments. However, single molecule resolution using similar magnetic technology requires a highly uniform particle size distribution.

Stoke's equation, which is provided below,

$$V = \frac{F}{6 πμ R}$$

relates the velocity V of a spherical particle of radius R in a solution of viscosity μ to the force F applied to that particle. From this equation it can be derived that in the presence of an applied magnetic force, differently sized magnetic particles move through a medium at different velocities. This concept can be used to separate differently sized magnetic particles and, importantly, also can be used to identify different analytes attached to the magnetic particles.

It is preferable that the population of each the different magnetic species exhibit either a unique hydrodynamic volume (or cross section) or at least be restricted to a narrow range of particle size distribution. Furthermore the relative hydrodynamic volumes of the different magnetic species in the sample should be sufficiently different from each other to permit ready separation and detection.

Importantly, the present invention provides reproducible methods where reagent quantities of the highly uniform, high permeability magnetic transducers can be produced. Both functionalized and non-functionalized transducers can be prepared using this method. The velocity of the particle can be used to determine its hydrodynamic size. For example spherical particles of radius 50 and 60 nm in water have velocities of approximately 30 and 25 μm/sec, respectively, under the influence of a 10⁻¹⁴ Newton force.

The relative separation of differently sized species in a selected medium is related to their different hydrodynamic radii. Two different species that have a greater relative difference in their hydrodynamic radii will also exhibit a greater difference with their relative mobility (or velocity) in the medium under the influence of the same magnetic force. Conversely, when the relative hydrodynamic volumes of the two different species are similar, the two species will also exhibit similar mobilities under the influence of the same magnetic force. For example, when a large magnetic transducer binds to a relative small analyte, the hydrodynamic radius of the resulting bound complex may not differ significantly from that of the large unbound or free transducer. The relative mobilities of the two species, i.e., the free and bound transducers, will be similar. The same phenomenon occurs in the situation when a relative small functionalized magnetic transducer is capable of binding to two different, but much larger analytes. In this situation the two different bound transducer complexes will exhibit similar mobilities in a magnetic field.

For some analytes, the binding agent used to functionalize the transducer is unique for that analyte. The properties and size of the binding agent may not be variable or may be variable to a very limited degree. Consequently, the binding agent for a particular analyte may not be varied to affect a different hydrodynamic radii for the bound transducer.

The present invention provides a method for the fabrication of magnetic transducers exhibiting a preselected or predetermined hydrodynamic volume as desired. The desired radius can be prepared according the present invention as discussed herein. Consequently, the functionalized transducer can be tailored for specific analytes or a target material.

The nanoparticle's magnetic moment also affects its mobility in a magnet field. The magnetic force applied to a superparamagnetic particles in an external field gradient is

$$F = {μ}_{o} XvH \frac{ⅆ H}{ⅆ x}$$

where μo is the permeability of free space, X is the susceptibility per volume of magnetic carrier, v is volume of magnetic carrier, and H is the magnetic field. The magnetic force is the other variable in Stoke's equation that can be used to modify the velocity of a particle. Careful design of the magnetic transducers, to control their magnetic susceptibility, for example, varying the Fe/Au atom ratio, will produce significant shifts in the magnetic force that could be used to amplify signal or enhance specificity. If multiple magnetic transducers are used detection could be multiplexed by varying both the hydrodynamic cross-section and the magnetic susceptibility of the different magnetic transducers to simultaneously identify multiple pathogens in a single sample.

If the magnetic transducer/target complex is large enough so that it can be optically tracked, the presence of target material in a sample can be conveniently detected using a microfabricated detection chamber in which well-defined electromagnetic fields are generated with integrated fluidics. A miniature optical tracking system based on a simple laser-detector system can be used to monitor the mobility of the transducers for separation and detection.

A wide variety of materials can be used as the fluid medium or matrix for magnetophoretic separation. Preferably the bound transducer complex should exhibit a mobility in the medium suitable for detection under the test conditions within a reasonable time frame. Preferably the media is selected to allow suitable separation in less than about 1 hour, more preferably in less than about 30 minutes and still yet more preferably less than about 15 minutes. Specific examples of preferred media for magnetophoretic separation include, but are not restricted to: water, agarose, (particularly, agarose diluted with water), and other materials known to from loosely crosslinked gel networks.

Synthesis of Nano-Composite Magnetic Transducer Particles

The present invention provides a method for the fabrication of nano-composite transducers exhibiting a preselected or predetermined hydrodynamic volume, magnetic moment, and optical signature as desired. Consequently, the functionalized transducer can be tailored for specific analytes or target material.

In one preferred embodiment of the present invention, DNA functionalized Fe/Au and Au nanoparticles can be chemically self-assembled into a composite transducer with single component particle resolution. The Fe/Au and Au nanoparticles are assembled in solution into composite transducers using complementary strands of DNA. The temperature, salt concentration, DNA coverage, relative particle concentration, and magnetic field can be used to control the size and geometry of the composite transducer. If an unwanted distribution in the size or shape of the transducer complex results, gel electrophoresis can be used to resolve the different size and shape complexes. The technique is not unique for DNA functionalized component particles. It will be understood that binding agents other than DNA can be used.

In yet another embodiment, magnetic transducers can be prepared by first fabricating a Fe/Au nanoparticle core of a desired size and magnetic moment. The Fe/Au nanoparticle core can be functionalized with a desired binding agent. The binding agent can be selected as desired. For example, either an antigen or its antibody can be used as the binding agent. In other embodiments, use of a DNA fragment is preferred.

Separately, Au nanoparticles are prepared. The Au nanoparticles are functionalized with a second agent selected to bind to the binding agent. For example, the Au nanoparticle can be functionalized with a complementary DNA fragment to that used to functionalize the Fe/Au nanoparticle. If necessary or desired for the particular application, the functionalized Au nanoparticles can be sized selected by size-selective precipitation or gel electrophoresis.

The functionalized Fe/Au nanoparticle core can be combined with an excess of the functionalized Au nanoparticles. When the binding group on the Fe/Au particle is a single stranded DNA fragment, the Au particle will include the complimentary single stranded DNA fragment. The two DNA strands will hybridize and serve as a crosslinking group for the Fe/Au and Au particles. The excess amount of the Au nanoparticle can be predetermined to provide a mixed metal composite that includes a Fe/Au nanoparticle as the core component surrounded or coated with a plurality of functionalized Au nanoparticles. The number of functionalized Au nanoparticles surrounding the Fe/Au core can be controlled by controlling the ratio of functionalized Au nanoparticles to Fe/Au core particles in solution.

It will be understood that for both the Fe/Au particles and the Au particles a plurality of binding groups can be attached to each particle. Consequently, each Fe/Au particle can bind to a number of different Au particles. The converse is also true, i.e., that a single Au particle can bind to a number of Fe/Au particles. Consequently a composite comprising a plurality of Fe/Au particles and a plurality of Au particles can grow in solution.

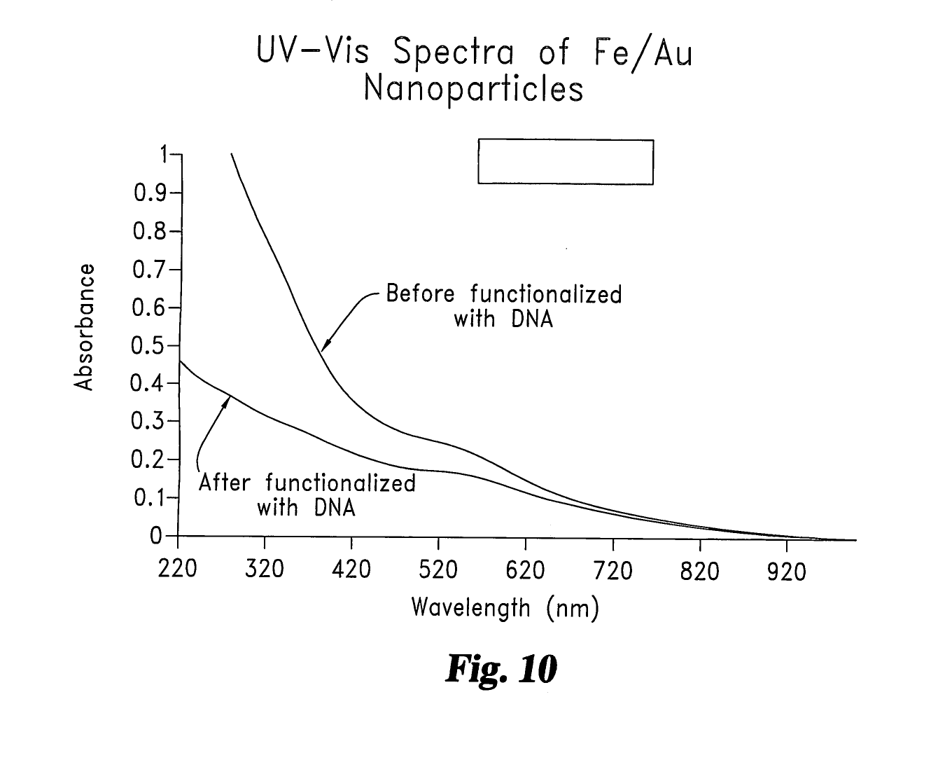

In still yet other embodiments, the functionalized Fe/Au and Au nanoparticles can be fabricated using different binding agents. A linker group can be used to crosslink the two binding agents, and consequently, form a mixed particle composite. This finds particular advantages when both the Fe/Au and Au nanoparticles are functionalized with single stranded DNA fragments. A third molecule can be used to link the two DNA strands together. This third molecule can be a target DNA fragment, another DNA linking group or nucleic acid fragment, as illustrated in FIGS. 10a, and b. In a preferred embodiment, the third molecule is a target DNA fragment, which is complimentary to at least a portion of the two DNA fragments attached to the two metal centers. In this embodiment, one end of the third DNA fragment hybridizes to at least a portion of the DNA fragment attached to the Fe/Au nanoparticle while the other end of the third DNA fragment hybridizes to a portion of the DNA fragment attached to the Au nanoparticle, thus linking the two nanoparticles.

Fabricating a composite nanoparticle as described herein can provide a highly controlled particle size, magnetic moment, and optical signature. Such composite particles are suitable for single molecule resolution using magnetophoresis. The size of a composite nanoparticle can be varied over a wider range than is easily obtained using the DACS. In selected embodiments, the particle size can be preselected to be between about 20 nm and about 200 nm. Although it will be understood that the particle sizes can be selected to be either smaller or larger than the above listed range.

FIG. 8 is a TEM micrograph of composite particles consisting of a magnetic Fe/Au core decorated on its surface with a small number of Au nanoparticles. These composite particles were self-assembled in solution using the methods described above from 10 nm diameter Fe/Au nanoparticles and 4 nm Au nanoparticles. The average diameter of these composite particles is approximately 150 nm.

Optical Detection of the Bound Transducer Complex

Bound transducer complexes can be detected in a number of different ways. Detection methods include, for example, detecting electron scattering density using transmission electron microscopy and detecting optical absorption using phase contrast imaging and video-enhanced contrast techniques. For example, transmission electron microscopy can be used to detect a bound transducer complex by detecting the constituent Au and/or Au/Fe particles (e.g. FIG. 8). This requires collecting a sample containing the bound transducer complex on a TEM grid. Single Au particles with diameters greater than about 20 nm can be detected optically. This is most easily done by collecting a sample on a glass substrate, as depth of field problems associated with particles in solution can make tracking their motion difficult. Single Au particles with diameters greater than about 50 or 100 nm can be optically detected in solution by using an optical microscope.

The Fe/Au particles or the pathogen species can also labeled with an optical marker such as a fluorescent molecule or a semiconductor nanoparticle quantum dot (J. Phys. Chem. B 101, 9463 (1997)), or with calorimetric, radioactive, chemiluminescent, electrochemiluminescent or enzymatically detectable agents. For example, detection can be accomplished using an immunological fluorometric assay, wherein an antigen attached to the Fe/Au nanoparticle reacts with an antibody carrying a fluorescent label. When an external labeling agent is utilized, it preferably labels the biological target rather than the Fe/Au nanoparticle. Use of labeling agent that labels the biological target allows the target to be detected without the need to separate the bound transducer complex from the unbound (free) magnetic transducers. Labeling of the Fe/Au nanoparticle is also envisioned, but in that event the bound transducer complex must be separated from unbound magnetic transducers prior to labeling.

In a particularly preferred embodiment, optical detection of the bound transducer complex is achieved through the use of an optical marker in the form of a bound Au nanoparticle. It is possible to detect single Au nanoparticles with diameters larger than approximately 20 nm by use of phase contrast imaging with a standard CCD camera (Biophysics. J. 52, 775 (1987)) and it is possible to functionalize the Au nanoparticles using the same methods as used for the Fe/Au nanoparticles.

In this embodiment of the detection method, a biological target is detected by contacting it with two different populations of metal nanoparticles: a population of Fe/Au nanoparticles having both controlled size and controlled Fe/Au ratio, which nanoparticles are functionalized so as to form complexes with the biological target of interest, and a population of Au nanoparticles also of controlled size that likewise complex with the biological target but do not complex with the Fe/Au nanoparticles (i.e., that do not exhibit nonspecific binding). Conditions favoring the formation of complexes between the nanoparticle reagents and the biological target are then established, followed by application of an external magnetic field to collect: 1) the Fe/Au particles that are not part of complexes, 2) the Fe/Au particle/biological target (bound transducer) complexes, and 3) the Fe/Au particle/biological target/Au particle (bound transducer) complexes. Because of the difference in the optical cross-sections of Au and the Fe/Au particles, it is possible to discriminate between the three species that are collected. As no Au particles that are not incorporated in a Fe/Au particle/biological target/Au particle complex will be collected, optical detection of an Au particle is proof of the presence of the biological target. This strategy may also work to optically detect bound complexes containing larger (e.g., micron-scale) magnetic particles, however it is also possible that nonspecific binding between the Au particle and the larger magnetic particle might occur, increasing the number of false positive results. Furthermore, when the Fe/Au particle/biological target complex is optically distinguishable from the Fe/Au particle/biological target/Au particle complex, this makes possible an embodiment wherein the Fe/Au nanoparticles are functionalized to bind to a broad class of biological targets, while the Au particles are functionalized with a different binding agent to bind a subset of the broad class, allowing detection of both the class of biological targets and selected members of the class.

To recapitulate, the combination of biospecific complexing of a biological target with the two kinds of nanoparticles (Fe/Au and Au) to yield a doubly bound transducer complex, magnetic harvesting of these complexes because of the magnetic Fe/Au clusters, and optical counting of the complex by counting the captured Au clusters enables rapid identification of individual biological species (targets). This scheme provides a highly sensitive and extremely low cost pathogen detection system.

This embodiment of the detection method of the invention does not necessarily depend on discrimination among magnetic transducer species based on their mobility in a magnetic field as long as separation between magnetic and non-magnetic species can be achieved and magnetic transducer complexes can be distinguished from free magnetic particles (as described above). However, in another embodiment of the method of the invention, because of precise control of the size and the magnetic susceptibility of Fe/Au nanoparticles allowed by the invention, detection could be based on measurement of magnetic carrier/biological target mobility in a magnetic field, i.e. magnetophoretic identification.

Detection of the Magnetic Moment of the Bound Transducer Complex

Selected bound complexes can be detected by measuring the magnetic moments of the unbound (free) transducer(s) and the bound transducer complex. The magnetic moments of the individual Fe/Au nanoparticles are affected by the magnetic moments of adjacent paramagnetic particles or groups. This interaction drops off rapidly the further apart the two paramagnetic centers are to each other.

In one form of the present invention, this interaction can be used to detect and identify target materials, particularly, target DNA fragments in the sample.

A single stranded DNA fragment can be modified or derivatized to include two sites capable of binding or functionalizing two Fe/Au nanoparticles. This can be accomplished, for example, by adding an excess of Fe/Au nanoparticles and an excess amount of the derivatizing species such as the HO—(CH₂)S—S(CH₂)₆— oligomers. The resulting DNA fragment has a Fe/Au nanoparticle attached to both its 5′ and 3′ ends. In solution, the single stranded DNA is flexible. Consequently, the two nanoparticles are not constrained in space relative to one another but are free to move relative to each other.

However, when the single DNA fragment hybridizes with a complimentary DNA strand, the resulting double stranded DNA is not as flexible as the single stranded DNA. This constrains the two nanoparticles in space. Once the two nanoparticles are constrained in space relative to each other the effect of the dipole-dipole interactions on the magnetic moments of each particle can be determined. The change in the magnetic moment of the Fe/Au nanoparticles of the single stranded DNA from that observed in the double stranded DNA can be used to detect the presence and relative concentration of the complimentary DNA strand in the sample. The present invention is illustrated by the following examples. It is to be understood that the particular examples, materials, amounts, and procedures are to be interpreted broadly in accordance with the scope and spirit of the invention as set forth herein.

### EXAMPLES

### Example I

### Distributed Arc Cluster Source (DACS) Operating Conditions for Synthesis of Au—Fe Nanoparticles

The total mass of metal placed in the DACS crucible was about 0.5 g with a known gold to iron weight ratio. The gold and iron used were 0.04 in diameter wires purchased from Alfa Aesar and were at least 99.9% pure. Argon was used as the inert gas in the arc chamber. The argon flow rate was 120 cm³/s at a pressure of 30 psig. Nitrogen or helium gas was used as the quench gas with a flow rate of 250 cm³/s or 425 cm³/s, respectively, at a pressure of 40 psig. Argon was allowed to flow through the apparatus for about 20 minutes prior and after a run. The gold-iron mixture in the crucible was heated with the plasma arc for five to ten seconds at an input voltage of 75% to pre-melt the feed before starting a run. This was done to homogenize the charge in the crucible. About 2-20% of the feed was evaporated during this pre-melt step.

To initiate the arc plasma, the variac was set at 75%. At this setting, the initial voltage drop between the tungsten electrode and the crucible was about 50V. Once the arc plasma formed, this voltage drop decreased to 16-20V. The variac was then decreased to 55-62% for the remainder of the run. At this variac setting, the voltage drop across the arc ranged from 11V to 14.5V, depending on the condition of the charge, the crucible, and the tungsten electrode. For instance, if the crucible is old with metal residues from previous runs or if the tungsten electrode is coated with evaporated metal, the voltage drop is usually higher. The voltage drop also increases with increasing distance between the tip of the tungsten electrode and the surface of the liquid pool in the crucible. For all the Au—Fe DACS runs in the present application, this distance was always set to be approximately 5 mm.

During the DACS run, the arc voltage and the arc current stayed quite stable. This indicated the presence of a stable plasma throughout the run. The arc current typically ranged from 56 A to 70 A and the arc power, which was estimated by the product of voltage drop and arc current, ranged from 630 W to 1040 W. The metal evaporation rate ranged from 4 mg/hr to 350 mg/hr. The evaporation rate does not necessarily increase with increasing arc power as expected. Clearly, there are other factors that govern the condition of the DACS plasma and the evaporation rate. The expected correlation between arc power and evaporation rate is based on the assumption that the product of arc voltage and arc current is a good measure of the energy supplied to the melt and thus of the melt temperature. However, this may not be the case. A large fraction of the plasma power is dissipated by radiation, and the arc does not always center on the crucible. Furthermore, large variations in arc voltage were observed at the same arc current, and the arc voltage does not necessarily increase with increasing applied current. This seems to indicate that the arc voltage is more dependent on the conditions within the melt or the arc.

Temperature measurement experiments done by others on a pure argon arc with tungsten/copper electrodes have shown that the temperature profile of a plasma arc does not vary significantly with small changes in arc power, and the arc has a temperature gradient such that the temperature is highest at the center of the arc near the cathode and decreases towards the anode and the outer periphery of the arc. It is speculated that the variation in DACS evaporation rate may be due to variations in the distribution of the melt in the crucible, i.e. whether the melted metal in the crucible is gathered at the center of the crucible or plated on the sides of the crucible. Both conditions were observed when the apparatus was cooled down after the pre-melt. It is not clear what causes these variations. The variation in DACS evaporation rate may also be due to variation in the alignment of the tungsten electrode. Although it is assumed that the arc is distributed evenly between the tungsten electrode and the melt in the crucible, this may not always be the case. If the tungsten electrode is slightly askew, the plasma may be centered on one side of the crucible, resulting in the melt not being heated uniformly. At times, the tantalum shield surrounding the crucible melted on one side, indicating an electrode misalignment. Thus, slight misalignment of the tungsten electrode can affect the uniformity of the arc and thereby the evaporation rate.

In cases of especially high evaporation rate (above 100 mg/hr), the plasma arc was most often unstable at low input current and the stable arc voltage was usually high (above 13V). This is consistent with experimental characteristics found when an element with high ionization potential such as nitrogen, hydrogen or carbon is introduced into an argon arc. In such cases, the temperature of the arc is higher than that of a plasma arc sustained solely by ionized metals with much lower ionization potentials. During the pre-melt of the DACS feed, the arc at times sputtered some carbon from the graphite crucible holder and coated the metal feed and tungsten crucible with a thin layer of carbon. The presence of carbon in the arc might have caused an increase in arc temperature and thus increased the evaporation rate.

Table 1 summarizes the average evaporation rates and arc powers for various Au/Fe feed compositions. The arc power needed to sustain the arc does not show any distinct correlation with the feed composition, however, the evaporation rate is seen to generally increase with increasing gold composition. There also seems to be a step increase in evaporation rate between feed compositions below and above 50/50%. Perhaps this is because gold has a higher ionization potential than iron. In the presence of a gold-rich feed, the plasma arc is predominantly sustained by ionized gold vapor and would have a higher temperature than a plasma arc sustained by an ionized vapor containing more iron ions. This effect of gold can be especially seen in the 80/20% Au/Fe runs, which has consistent high arc voltages.

TABLE 1
Average evaporation rates and arc powers
for various Au/Fe feed compositions.

| Molar Feed | Average | Average | Average |
|---|---|---|---|
| Ratio | Evaporation Rate | Evaporation Rate | Power |
| (Au/Fe %) | (mg/hr) | (mol/hr) | (W) |
| 10/90% | 37.5 | 5.15E−04 | 775.12 |
| | 32.0 | 3.54E−04 | 841.07 |
| 50/50% | 76.1 | 6.68E−04 | 753.82 |
| 60/40% | 121.4 | 1.02E−03 | 760.26 |
| 70/30% | 132.2 | 1.06E−03 | 727.27 |
| 80/20% | 142.1 | 1.07E−03 | 811.62 |

### Example II

### Sample Preparation and Analytical Methods Used to Characterize Au—Fe Nanoparticles

The average composition of a sample of Au—Fe nanoparticles was determined using a Perkin Elmer AAnalyst 300 Atomic Absorption (AA) Spectrometer. This instrument determines the analyte concentration by measuring the amount of light absorbed by the analyte ground state atoms. Since each element only absorbs light energy of a specific wavelength, each element has its own specific AA operating conditions. The gold concentration was determined using a gold hollow cathode lamp (Fisher Scientific) at a wavelength of 242.8 nm, a slit width of 0.7 nm, and an input current of 8 mA (80% of the rated maximum current). The iron concentration was determined using an iron hollow cathode lamp at a wavelength of 248.3 nm, a slit width of 0.2 nm, and an input current of 24 mA. For each analysis, the spectrometer was calibrated with two to three samples diluted from AA standard solutions (Alfa Aesar). The gold standards used for calibration and the sample gold concentration typically ranged from 0 to 20 ppm, which is within the operating linear range for gold (0-50 ppm). For iron, the standard and sample concentrations were kept within the linear range of 0-10 ppm. The AA flame used for both gold and iron analysis was a lean blue air-acetylene flame. The recorded AA concentration was an average of five replicated readings taken is apart.

The morphology, homogeneity, and size of the nanoparticles were examined using a JEOL 2000FX Transmission Electron Microscope (TEM). The operating electron energy was at 200 keV. The TEM micrographs were taken at a magnification of ×100-600 k using a digital camera operated by the Gatan Digital Micrograph software. The TEM samples were prepared on carbon coated copper grids of 200 mesh purchased from Electron Microscopy Sciences. The size distribution of the nanoparticles was determined from the TEM micrographs using Optimas 6.1 software and Image Tool software.

Magnetic properties of the nanoparticles were determined at Carnegie Mellon University by Dorothy Farrell working in the laboratory of Professor Sarah Majetich. A Quantum Design MPMS SQUID Magnetometer was used. The magnetic measurements were taken at 100K and 293K.

A: Atomic Absorption Spectroscopy (AAS) Analysis

Nanoparticles captured with organic surfactants can be separated from the capture solution by mixing a polar organic solvent such as acetonitrile or ethanol with the non-polar capture solvent to reduce the steric repulsion between the surfactant encapsulated nanoparticles. The Au—Fe nanoparticles were separated from the mesitylene capture solution by mixing equal volumes of acetonitrile [CH₃CN] and the nanoparticle solution. After about an hour, the mixture was centrifuged for 60 minutes to segregate out the nanoparticles, which deposited as black or brown solids at the bottom of the centrifuge tube. The precipitated Au—Fe nanoparticles were then dissolved in 1.0 ml of aqua regia diluted with 30 ml of deionized water. (Aqua regia was prepared by mixing 3 parts by volume of hydrochloric acid with 1 part nitric acid. All acids were obtained from Mallinckrodt and were at industrial strength.) However, acetonitrile also caused precipitation of some of the surfactant not absorbed on the nanoparticles. The precipitated surfactant that did not dissolve in the acid was filtered from the solution or removed by centrifugation. The filtration method was found to be a more efficient way of removing the surfactants and yielded more accurate results than the centrifugation method. The AA sample solutions have to be solid-free to prevent clogging of the spectrometer tubing. The acid content within the AA sample preferably does not exceed 5% by volume, which is the recommended maximum acid tolerance for the AA spectrometer.

Composition of Au—Fe nanoparticles used for magnetic measurements was determined by separating the nanoparticles from the capture solution with a permanent magnet (see later discussion) and dissolving a small amount of the dried nanoparticles in 1 ml of aqua regia diluted with 30 ml of deionized water. Magnetic separation of the particles managed to separate the nanoparticles from excess surfactant. Therefore, these samples did not have problems with undissolved surfactant, allowing cleaner dissolution of the particles as compared to the samples prepared by the acetonitrile precipitation method.

Au—Fe nanoparticles captured in water were simply dissolved by adding 1.0 ml of the nanoparticle solution to 1.0 ml of aqua regia diluted with 30 ml of deionized water.

B: Transmission Electronic Microscopy (TEM) Analysis

TEM samples of organic solution captured nanoparticles can be prepared by casting a drop of the nanoparticle solution onto a TEM grid and slowly evaporating the solvent (Method 1). However, solvent evaporation does not remove excess surfactant from the TEM grid, as the surfactants are not volatile. Excess surfactants on the grid cause poor particle resolution and can oxidize or pyrolize in the electron microscope and hinder imaging. For accurate TEM imaging, the organic captured Au—Fe nanoparticles often had to be separated from the capture solution to remove excess surfactant. This was done by adding acetonitrile to the particle solution to precipitate the nanoparticles as described earlier. The precipitated nanoparticles were resuspended in 1 ml of dichloromethane under ultrasonication. Dichloromethane was used as opposed to mesitylene because it is much more volatile than mesitylene and facilitates the TEM sample preparation. The Au—Fe nanoparticles in dichloromethane were spread over a water surface framed with hexane. The hexane ring generally prevents the dichloromethane from sinking into the water phase as it has a higher density than water. The dichloromethane was allowed to evaporate and leave an array of nanoparticles on the water surface. The nanoparticle array was then transferred to a TEM grid by lightly touching the carbon coated copper grid on the water surface (Method 2).

TEM samples of Au—Fe nanoparticles in aqueous solution can be prepared by placing a drop of the particle solution onto the TEM grid and letting it dry in air (Method 3). This method, however, often results in the nanoparticles aggregating together as the water evaporates from the grid. Therefore, other methods were investigated to improve the quality of the sample. One of the methods used was mixing 100 μL of particle solution with 100 μL of tetrahydrofuran [C₄H₈O], placing the drop on a piece of Teflon, and heating it with a heat lamp (Method 4). As the drop evaporated, the nanoparticles were brought to the drop surface and formed a monolayer of particles on the surface. The nanoparticles were transferred onto the TEM copper grid by touching the grid on the drop surface. Another method was to cast a drop of the particle solution onto a TEM grid placed on a permanent magnet (Method 5). As the nanoparticles are magnetic, their magnetic moment causes them to be attracted to the magnet and to form chains of particles instead of dense aggregates. TEM analysis, however, showed that nanoparticle samples prepared by Method 4 and 5 do not significantly improve the dispersion or reduce the aggregation of the nanoparticles on the TEM grid as compared to samples prepared by Method 3.

C: Squid Magnetic Measurement

Au—Fe nanoparticles in organic solution were flowed slowly through a straw placed between the poles of a permanent magnet. The magnetic Au—Fe nanoparticles were deposited on the walls of the straw where the magnet was located. Nanoparticles that were extremely small or that had low magnetic moment bypassed the magnet and were captured in a flask. The nanoparticles deposited in the straw were dried on a petri dish and embedded in epoxy before being inserted into a clean straw for magnetic measurements.

The Au—Fe nanoparticles captured in water solution were first transferred into organic solution before being captured in the straw as described above. To transfer the charged stabilized nanoparticles into organic solution, 30 ml of the aqueous solution containing Au—Fe nanoparticles was added to 20 ml of ethanol and stirred for 2 minutes. A surfactant solution of 0.05M dodecanethiol, 0.02M dodecylamine, and 0.03M dodecylamine in ethanol was prepared. 2 ml of the surfactant solution was added to the particle solution, and the mixture was stirred for 20 minutes. The nanoparticles encapsulated by the organic surfactants were separated from the solution by centrifugation and re-suspended in mesitylene under ultrasonication.

### Example III

### Stabilization of DACS Au—Fe Nanoparticles in Organic and Aqueous Solutions

This example describes experiments using different stabilizing agents to encapsulate Au—Fe nanoparticles in organic and aqueous solutions. Mesitylene (1,3,5-trimethylbezene), a non-polar solvent, was used as the organic solvent. The mesitylene used was purchased from Aldrich and had 97% purity. In mesitylene, oleic acid [CH₃(CH₂)₇CH═CH(CH₂)₇CO₂H], 1-dodecanethiol [C₁₂H₂₅SH], didecylamine [C₁₂H₂₅NH₂], and didecylamine [(C₁₀H₂₁)₂NH] were used as stabilizing surfactants. The organic surfactants were purchased from Aldrich and had 98% purity. Oleic acid was used by itself and was prepared by adding 0.282 g (1 mmol) of oleic acid into 120 ml of mesitylene. The thiol and amine surfactants were used both by themselves and as mixtures in mesitylene. The usual amounts of dodecanethiol, dodecylamine, and didecylamine used were 1.0 ml (4.2 mmol), 0.05 g (0.27 mmol), and 0.05 g (0.17 mmol), respectively in 120 ml of mesitylene.

Citric Acid [HOC(CO₂H)(CH₂CO₂H)₂], sodium citrate [HOC(CO2⁻Na⁺)(CH₂CO₂Na⁺)₂], Bis(p-sulfonatophenyl)phenyl phosphine dipotassium salt [C₆H₅P(C₆H₄SO₃⁻K⁺)₂], and methoxy polyethylene glycol-sulfhydryl [CH₃—(OCH₂CH₂)ₙ—SH] were used to stabilize the Au—Fe nanoparticles in water. These chemicals were purchased from Aldrich, Mallinckrodt, Strem Chemical, and SunBio PEG-Shop, respectively, and had 99% purity. The usual amounts used were 0.31 g (1.61 mmol) for citric acid, 0.04 g (0.17 mmol) for sodium citrate, 0.1 g (0.2 mmol) for phenyl phosphine, and 1.16 g (0.58 mmol) for polyethylene glycol in 120 ml of water.

A: Au—Fe Nanoparticles Captured with Oleic Acid in Mesitylene

The first organic surfactant used to capture the Au—Fe nanoparticles in organic solution was oleic acid. Oleic acid was chosen to capture the Au—Fe nanoparticles because it has been known to successfully stabilize silver particles in organic solution, and the surface properties of silver is quite similar to gold. The long carbon chain of oleic acid makes it soluble in organic solvents, while its polar carboxylic acid end attaches to the surface of the Au—Fe nanoparticles. The Au—Fe nanoparticles formed a metastable colloid in oleic/mesitylene solution and had a faint pinkish color. From TEM micrographs of 50/50% Au/Fe feed ratio nanoparticles captured with oleic acid in mesitylene, the particles appear to have an average size of 10 nm. It is also apparent that excess oleic acid remains on the TEM grid once the mesitylene evaporated.

Oleic acid captured nanoparticles could not be easily re-suspended in organic solvent once they had been centrifuged from a mixture of capture solution and acetonitrile. This is believed to be due to the fact that the oleic acid molecule is not strongly bonded to the metal particles and can be easily displaced. The problems encountered with oleic acid led to trials of other organic surfactants to capture Au—Fe nanoparticles.

B: Au—Fe Nanoparticles Captured with Thiol Surfactant in Mesitylene

A DACS run with a 50/50% Au/Fe feed composition was performed with a dodecanethiol/mesitylene capture solution. Dodecanethiol is known to bind strongly to gold surfaces, and thus was chosen to stabilize the Au—Fe nanoparticles. The Au—Fe nanoparticles suspended as metastable particles in thiol/mesitylene and formed a brownish solution. TEM micrographs were made of the Au—Fe nanoparticles captured with dodecanethiol surfactant in mesitylene. The nanoparticles are not uniform in size. The big nanoparticles might have formed during the DACS startup when the evaporation rate is higher. Big nanoparticles may also form in the gas phase or in the capture solution due to particle aggregation and flocculation before they can be encapsulated by the surfactants. On average, the thiol-encapsulated nanoparticles initially had an approximate size of 6 nm. However, the nanoparticles appeared to be unstable and grew in size after a couple of days in the capture solution. After about 20 days, the particles have grown to about an average size of 10 nm. This particle growth may due to the weak bonding of the alkanethiol on surface iron atoms. This results in the formation of a defective SAM layer or partial coverage of the nanoparticles by the surfactant. Defects in the SAM layer coating the particles provide sites for particle growth or aggregation.

C: Au—Fe Nanoparticles Captured with Amine Surfactants in Mesitylene

A DACS run with 50/50% Au/Fe feed was performed with a mixture of dodecylamine and didecylamine surfactants in mesitylene. The amine surfactants were used because alkyl amines are known to bind on iron surfaces. The amines are expected to only bind weakly on gold surfaces. The Au—Fe nanoparticles suspended as metastable particles in the aminelmesitylene solution and formed a brownish solution. The Au—Fe nanoparticles captured with amine surfactants have an average size of 13 nm and are highly uniform in size compared to the dodecanethiol-captured nanoparticles. However, these amine-captured nanoparticles tend to flocculate and form nanoparticle aggregates.

D: Au—Fe Nanoparticles Captured with a Mixture of Thiol and Amine Surfactants in Mesitylene

The Au—Fe nanoparticles from a DACS run with 50/50% Au/Fe feed composition captured with a mixture of dodecanethiol, dodecylamine, and didecylamine surfactants in mesitylene formed a brownish solution. The mixed surfactant captured Au—Fe nanoparticles have a fairly wide size distribution, which typically ranges from 5 to 50 nm. The average particle size is estimated to be 10 nm. These nanoparticles are much more stable than the nanoparticles captured with either thiol or amine surfactants alone. The presence of acetonitrile tends to reduce the steric repulsion and causes the particles to flocculate. However, the Au—Fe nanoparticles do not appear to have aggregated or grown in size. The average particle size is still 10 nm. The stability of these nanoparticles is thought to be due to the effective coverage of the nanoparticles with surfactants that have great affinity towards both gold and iron surface atoms. The amine surfactants are expected to bind strongly to the iron surface atoms and the thiol surfactant to the gold surface atoms.

Of the organic solutions examined, the mixed surfactant solution with both thiol and amine surfactants was found to be the most effective capture solution for the DACS synthesized Au—Fe nanoparticles. The Au—Fe nanoparticles appeared to be most stable in this solution and could be easily resuspended in clean (surfactant-free) organic solution even after being centrifuged from the original capture solution. This mixed surfactant solution was therefore used to capture all the Au—Fe nanoparticles samples sent for magnetic analysis of the organic captured DACS nanoparticles.

E: Au—Fe Nanoparticles Stabilized with Citric Acid in Water

Citric acid was first used as a water-soluble capture agent because citrate ion is used as the stabilizing agent for commercially available Au colloids. Au—Fe nanoparticles captured using citric acid formed a slightly pink solution and were very uniform in size. The Au—Fe nanoparticles from a 50/50% Au/Fe feed composition DACS run captured in a citric acid/water solution have an average size of 10 nm. However, after one day in the solution, most of the particles settled out of the solution and the capture solution became greenish in color. It is suspected that the iron atoms were leached from the particles and formed Fe(III), which is green in color when dissolved in water. AA analysis on the aqueous solution of nanoparticles captured using citric acid yielded a constant iron composition of 99% regardless of the variation in the DACS feed iron composition from 40-70%. It is felt that the nanoparticles had largely precipitated, leaving a solution containing mainly of dissolved iron. To test this hypothesis, the precipitated particles of a 30/70% Au/Fe DACS sample were analyzed by AA and were found to have a composition of 23% Au and 77% Fe while the composition obtained for the bulk solution containing the suspended “particles” was 1% Au and 99% Fe. In a second experiment, Au—Fe nanoparticles from a 50/50% Au/Fe DACS run that were sampled by dissolving the particles that had deposited on the plastic tubing leading from the DACS to the capture vessel were found to have a composition of 41% Au and 59% Fe while the composition of the citric acid “colloidal” solution from the same run had a composition of 10% Au and 90% Fe. Thus, citric acid is not an effective capture agent for Au—Fe nanoparticles in water.

F: Au—Fe Nanoparticles Stabilized with Bis(p-sulfonatophenyl)Phenyl Phosphine Dipotassium Salt in Water

This phosphine compound is known to stabilize Au particles in water. The phenyl groups attached to the phosphorous atom are functionalized with sulfates, which are negatively charged, and impart charge stabilization to Au nanoparticles.

The Au—Fe nanoparticles suspended in the phosphine/water capture solution and formed a brownish solution. The Au—Fe nanoparticles captured with phosphine in water from a 50/50% Au/Fe feed composition DACS run have a size range of 3-25 nm and an average particle size of 8 nm.

G: Au—Fe Nanoparticles Stabilized with Sodium Citrate in Water

The citrate ion has three carboxylic groups, which become negatively charged when dissolved in water. The citrate ions will therefore be drawn towards positively charged metal particles in water and form an electrical double layer around the particles. Citrate is known to stabilize Au particles in water.

The Au—Fe nanoparticles suspended in citrate/water capture solution and formed a brownish solution. The Au—Fe nanoparticles from a 50/50% Au/Fe DACS run stabilized by citrate in water had a size range of 3-20nm and an average particle size of 6 nm.

H: Au—Fe Nanoparticles Stabilized with Methoxy Polyethylene Glycol Sulflydryl (PEG-SH) in Water

For many biological applications, it is desirable to produce Au—Fe nanoparticles that are stabilized by a water-soluble molecule that is covalently bonded to the particles. PEG-SH is a molecule with a thiol head group, which has great affinity towards gold atoms, and an ethylene glycol chain, which makes it soluble in water. Au—Fe nanoparticles captured using PEG-SH in water formed a brownish solution. The Au—Fe nanoparticles of a 50/50% Au/Fe DACS run captured by the PEG-SH in water have diameters ranging from 5 to 50 nm with an average size of 16 nm. The PEG-SH captured nanoparticles have an average size larger than those captured with either organic surfactants or phosphine and citrate ions. There is a likelihood that the PEG-SH is not able to attach to the particles quick enough to prevent particle aggregation in solution. In addition, the PEG-SH did not generally impart long-term stability to the nanoparticles. After two days, the solution lost its brown color and a large amount of yellow precipitate was found. TEM analysis of a sample of the solution revealed no observable particles. The yellow precipitates were checked for magnetism with a permanent magnet and were found to be not magnetic. It is believed that these precipitates largely consist of polymerized PEG-SH. It appears that the PEG-SH molecule is not able to efficiently capture and stabilize the Au—Fe nanoparticles in water.

G: Phase Transfer of Au—Fe Nanoparticles from an Aqueous Solution to an Organic Solution

The Au—Fe nanoparticles captured in water were transferred into organic solution for preparation of magnetic measurement samples. This is to ensure that the nanoparticles do not aggregate and grow when they are separated out from solution by the magnet and dried prior to encapsulation in epoxy. Water-soluble stabilizing agents such as sodium citrate, which stabilize the particles by charge, lose their ability to prevent particle aggregation once the particles are not in solution. On the other hand, organic surfactants such as alkyl thiol and alkyl amine, which stabilize the particles by steric repulsion, form a SAM layer that is bonded to the particle surface and can thus prevent particle aggregation when the particles are not in solution. Phosphine stabilized and citrate stabilized nanoparticles were encapsulated by a mixture of thiol and amine surfactants with the procedure herein and examined for any changes in their physical properties.

Phosphine stabilized Au—Fe nanoparticles can be encapsulated with organic surfactants without any significant change in particle size. The Au—Fe nanoparticles (50/50% Au/Fe DACS feed) did not grow in size after being encapsulated by thiol and amine surfactants. The average particle size was 7 nm before and after the transfer. AA analysis of the particle composition before and after the transfer also showed that the particle composition did not change significantly. The average particle composition in phosphine solution was 45% Au and 55% Fe while the average particle composition in organic solution was 46% Au and 54% Fe. Therefore, phase transfer of phosphine stabilized Au—Fe nanoparticles into organic solution does not significantly change the size distribution or average composition of the nanoparticles.

The citrate stabilized Au—Fe nanoparticles can also be encapsulated with organic surfactants without significant changes in size or composition. Evaluation of the size distributions before and after encapsulating citrate stabilized Au—Fe nanoparticles with mixed thiol and amine surfactants showed that the nanoparticles retained their average particle diameter of 6 nm after the transfer. AA analysis on these Au—Fe nanoparticles showed that the average particle composition in the citrate solution was 46% Au and 54% Fe, and the average particle composition in the organic solution was 44% Au and 56% Fe. This slight difference in the particle composition may be due to the difficulty in determining an accurate gold composition in the particles captured using thiol surfactant. Thus, the particle properties are assumed to be unchanged during the process of transferring the citrate stabilized particles into organic solution.

H: Narrowing the Size Distribution of Au—Fe Nanoparticles

DACS synthesized Au—Fe nanoparticles captured in organic solution using mixed thiol and amine surfactants usually have a fairly wide size distribution. To improve the uniformity of the particle size, the Au—Fe nanoparticles stabilized by mixed thiol/amine surfactants can be selectively precipitated using acetonitrile. By adding a small amount of acetonitrile to the particle sample, the larger nanoparticles can be induced to flocculate and can then be removed from the solution by centrifugation while the smaller nanoparticles remain in solution. To size select the Au—Fe nanoparticles, a DACS nanoparticle sample was allowed to sit for a day to allow the largest nanoparticles to settle out of the capture solution. To a 4.8 ml sample of the colloidal suspension was added 1.2 ml of acetonitrile (20volume %). The mixture was allowed to sit for 90 minutes before centrifuging it for 60 minutes. The precipitated particles, which looked black, were discarded, and to the remaining solution, which contained unprecipitated particles, was added with an additional 3.6 ml of acetonitrile (50volume %). The mixture was allowed to sit for one hour before centrifuging it for another hour. The precipitated nanoparticles, which looked like a light brown solid, were allowed to dry. These dried nanoparticles were then resuspended in 1 ml of dichloromethane under ultrasonication to yield a brown suspension. The nanoparticles have a size range of 4 to 30 nm and an average size of 10 nm before size separation, and a tighter size range of 4 to 10 nm and an average size of 5 nm after size separation. Thus, the size distribution of the Au—Fe nanoparticles captured using the mixed thiol/amine surfactants can be improved by selective precipitation.

Citrate stabilized Au—Fe nanoparticles can be encapsulated with the mixed thiol/amine surfactants and transferred into mesitylene before being size selected by acetonitrile precipitation. The citrate stabilized Au—Fe nanoparticles were recaptured in organic with the procedure described herein, and size selected with the same procedure described above. However, in this case, the nanoparticles recaptured in organic solution from a citrate/water solution were size selected using 5 volume % acetonitrile instead of 20%. Before size selection, the nanoparticles had a size range of 3-12 nm and an average size of 6 nm. After size selection, the nanoparticles were very uniform in size with an average particle size of 5 nm. A direct procedure has yet to be found to successfully improve the size distribution of Au—Fe nanoparticles in aqueous solution without first transferring the particles into organic solution.

### Example IV

### TEM Analysis of the Structure of Au—Fe Nanoparticles

The DACS synthesized Au—Fe nanoparticles with feed compositions ranging between 30/70% and 80/20% Au/Fe were found by TEM analysis to exhibit no obvious segregation of the iron and gold atoms. It was found that the Au—Fe nanoparticles usually exhibit an even intensity across the particle image, implying that the particle density is uniform and that there is a uniform distribution of gold and iron atoms within the particles. The bigger particles are darker than the smaller particles due to the difference in electron scattering from particles of different thickness. However, particles of similar size also exhibit different intensities. This may be due to an uneven distribution of gold and iron among the particles or it may be due to difference in the orientation of these particles relative to the electron beam. Since gold has a higher atomic number than iron, it has a larger cross-section electron scattering than does iron, thus particles that are richer in gold are expected to look darker than the particles richer in iron. A few of the Au—Fe nanoparticles have different intensities within the particle itself, such as a dark ring surrounding a lighter core or a dark hemisphere attached to a lighter hemisphere. This is most probably due to formation of gold-rich and iron-rich phases within the particles.

For nanoparticles synthesized with feed composition above 70% Fe, a core-shell heterogeneous structure is observed. Since gold has a higher surface free energy than iron, most of the particles from a 10/90% Au/Fe feed run captured in citrate/water solution have a core-shell structure with a lighter iron-rich layer surrounding a darker gold-rich core. AA analysis of these heterogeneous particles showed that the particles have a composition of 12% Au and 88% Fe. Formation of core-shell heterogeneous particles is expected for Au/Fe compositions above 30/70% based on the Fe/Au binary phase diagram. Above this composition limit, an iron-rich phase is expected to precipitate first from a homogeneous liquid phase as the particle cools. Further cooling leads to formation of the gold-rich phase.

In conclusion, the TEM analysis indicates that DACS synthesized Au—Fe nanoparticles are single phase, i.e. homogeneous as long as the iron composition is less than ˜70% although they are not necessarily uniform in size or composition.

### Example V

### Correlation Between the Composition of DACS Synthesized Particles and the Composition of the DACS Feed

The composition of DACS particles was investigated to examine how the particle composition varies with the composition of the DACS feed. By manipulating the Au/Fe ratio, the magnetic moment of the Au—Fe nanoparticles can be controlled independently of particle size.

The evaporation in the DACS occurs at a very high temperature. Therefore, it is speculated that the partial pressures of Au and Fe vapor in the arc can be modeled using Raoult's Law, which states that the partial pressure of a component in an ideal system is equal to the product of its liquid phase composition and its pure vapor pressure. As the pure vapor pressures of Au and Fe are almost identical at high temperatures (VP_{Fe}/VP_{Au}=0.95 at ˜3500K), it is expected that the evaporation rates of Au and Fe in the DACS should be approximately proportional to their relative compositions in the melt.

It can be seen from an analysis of gold atomic fraction in the DACS nanoparticles relative to the gold atomic fraction in the feed that the particle composition generally tracks the feed composition. However, there is a lot of scatter in the data. In particular, the composition of nanoparticles captured in organic solution does not appear to correlate well with the feed composition. For example, when the DACS feed composition is 50/50% Au/Fe, the average composition of the particles captured using the thiol surfactant alone is 33% Au and 67% Fe. However, with the same DACS feed, the composition of particles captured using the amine surfactant alone is 45% Au and 55% Fe. When the Au—Fe nanoparticles are separated from solution by adding acetonitrile and centrifuging, much of the excess surfactant also precipitates with the particles. As a result, when aqua regia is added to dissolve the clusters, white undissolved solids appear in the acid solution. The undissolved solids were removed by centrifuging or filtering the solution. However, the presence of excess surfactant appears to affect the analysis of the particle composition.

To investigate whether the thiol surfactant can remove gold from an acidic solution, an experiment was performed in which a small amount of dodecanethiol was added to a dilute solution of known gold concentration. When the dodecanethiol was added to the gold solution, it formed an immiscible layer on top of the aqueous solution. After a few hours, this dodecanethiol layer turned slightly red while the aqueous phase turned from bright yellow to light yellow. When aqua regia was added to the two-phase mixture, white solids appeared and the organic layer was no longer present. The white solids were removed from the solution by centrifugation, and the aqueous phase was checked for its gold concentration. AA analysis of the aqueous phase showed that the gold concentration was reduced by 55%. Therefore, the presence of excess dodecanethiol when the nanoparticles are dissolved in aqua regia prevents accurate analysis of the composition of the nanoparticles.

In order to test whether the amine surfactant also interferes with the composition analysis, a small amount of the mixed amine surfactant was added to a known mixture of iron and gold standard solutions containing aqua regia. The mixture was allowed to sit for a few days after which the amine surfactants were removed from the aqueous phase. The aqueous phase was analyzed by AA, and in this case, the gold and iron concentrations were found to decrease by only 6%, which could be due to experimental error. Therefore, the presence of amine surfactant probably does not interfere with the dissolution of Au—Fe particles in aqua regia.

It is speculated that the surfactant interference problem can be solved by filtering the undissolved solids from the acidic solution and then rinsing them thoroughly with deionized water to remove any retained metal atoms, or by repetitive precipitation and resuspension of the nanoparticles in fresh solvent to remove the excess surfactant before dissolving the nanoparticles with aqua regia. An AA sample of 50/50% Au/Fe feed composition nanoparticles captured in thiol-amine solution was prepared with the filtration and washing procedure. The composition of the nanoparticles was found to improve significantly, yielding a composition of 42% Au and 58% Fe. AA analysis of Au—Fe nanoparticles separated from the organic capture solution using a magnet should also give reliable particle compositions. Magnetic separation of the particles is able to separate the particles from excess surfactants and no undissolved solids are seen when the dried particles are dissolved in acid solution.

It was further found that particle composition of the phosphine captured and citrate captured nanoparticles varies linearly with the feed composition. Unlike the situation with organic captured nanoparticles, there is no surfactant residue present when the nanoparticles captured in water are dissolved with aqua regia. However, the Au—Fe nanoparticle composition is not always the same for the same feed composition. This may be caused by a shift in the actual feed composition in the crucible due to reusing crucibles with leftover feed from previous runs. It is observed that DACS runs using old crucibles tend to yield particles that are richer in gold for the same Au/Fe feed composition. This suggests that there could be some iron-rich residue in the reused crucible, which could have lowered the arc temperature and shifted the equilibrium state towards forming particles with higher gold fraction. This composition variation may also be caused by variation in the condition of the generated plasma arc and thus the temperature of the arc.

The particles have higher Fe compositions than predicted by Raoult's law at 3000K. The Raoult's law prediction of particle composition calculated at temperatures 4000K and above seems to correlate better with the experimental results. There is a possibility that the actual arc temperatures are higher than expected. It is also plausible that the arc temperature changes with the composition in the melt, i.e. increases with increasing gold composition. Therefore, the Au—Fe particle compositions across the composition range may not be correlated by Raoult's law calculated at only one temperature.

The particle composition also seems to depend on the purity of the feed. Runs with only 99+% pure iron were found to have higher gold fractions than expected. It is speculated that somehow the iron purity affects the partial pressure of iron in the arc and decreases the iron composition in these particles. Iron less than 99.9% pure may have relatively high amount of impurities such as oxides, silicon, cobalt, or nickel, which could potentially decrease iron solubility in gold and the vapor pressure of iron.

### Example VI

### Magnetic Properties of DACS Synthesized Au—Fe Nanoparticles

Fe nanoparticles synthesized using a multiple expansion cluster source (MECS) and captured with organic surfactants were found to oxidize to α-Fe₂O₃ (rust) and lose their magnetic properties after a few hours in solution. The Au—Fe nanoparticles synthesized in the present examples, however, retain their magnetic properties after several months in solution whether they are captured in organic or aqueous solution. A coarse check on the magnetization of DACS synthesized Au—Fe nanoparticles can be done by placing a permanent magnet on the side of the sample bottle to see if the particles respond to the magnet. TEM analysis has shown that the Au and Fe atoms in the DACS nanoparticles do not phase segregate into obvious Au-rich and Fe-rich phases. Therefore, it is speculated that the iron atoms are isolated in the core of the particles and protected by Au from oxidation. In order to quantify the magnetization of the Au—Fe nanoparticles, the magnetic characteristics of the DACS nanoparticles were measured using the SQUID magnetometer in Professor Majetich's laboratory at Carnegie Mellon University.

A: Magnetization of Organic Captured and Aqueous Captured Au—Fe Nanoparticles

Magnetic measurements on the Au—Fe nanoparticles captured in organic solution using the mixed thiol-amine surfactants and in water solution using sodium citrate show that they are superparamagnetic with very small coercivity and remanence. The Au—Fe nanoparticles also exhibit a relatively large saturation magnetization. Magnetization curves were made of a sample of Au—Fe nanoparticles captured in organic solution with an average particle composition of 48/52% Au/Fe and a sample of Au—Fe nanoparticles captured in water solution with an average particle composition of 44/56% Au/Fe. (The Au—Fe nanoparticles stabilized by citrate in water were transferred into organic solution before being captured for magnetic measurements.) The magnetic measurements were performed at 100K and 293K within the magnetic field (H) range of ±50,000 Oe. As expected, the magnetization of the particles is higher at lower temperature. It was found that the nanoparticles initially captured in water have a lower magnetization than the nanoparticles captured in organic solution.

Table 2 summarizes the magnetic and physical properties of Au—Fe nanoparticles captured in organic solution, and Table 3 summarizes the magnetic and physical properties of Au—Fe nanoparticles captured in citrate solution. The small particle sizes of water-captured nanoparticles might be the reason for the lower saturation magnetization of water-captured nanoparticles as compared to organic captured nanoparticles. At a given composition, the fraction of iron atoms on the particle surface of a small particle is higher than that of a bigger particle. Since the nanoparticles captured in water are mostly below 8 nm in diameter, the ratio of surface iron atoms to core iron atoms is expected to be significantly high for these particles. As the surface iron atoms are predicted to be mostly oxidized, the ratio of oxidized iron atoms to unoxidized iron atoms within the small particles captured in water would be expected to be greater than that in the larger particles captured in organic solution.

Based on the saturation magnetization values measured in these experiments, the sample weight, and the particle composition, the magnetic moment per iron atom of the nanoparticles was calculated and plotted with respect to the average atomic fraction of iron in the particles. The saturation magnetic moment of the organic captured Au—Fe nanoparticles is roughly proportional to the iron atomic fraction within the particles. However, the magnetic moment per iron atom increases with increasing atomic fraction of iron instead of staying constant. Perhaps, in an iron-rich particle, the iron atoms coalesce into small atomic clusters, which yield a higher average spin moment. In a gold-rich particle, the iron atoms may be more highly dispersed among the gold atoms, thus lowering the average spin moment per iron atom.

Unlike the organic captured Au—Fe nanoparticles, the magnetic moment per iron atom of the water captured Au—Fe nanoparticles seems to decrease with increasing atomic fraction of Fe. This decrease may be caused by the fact that the water-captured nanoparticles are smaller in size than the organic captured nanoparticles and are therefore more sensitive to oxidation. Although sample A in Table 3 had a slightly higher average particle size than sample B, the iron atomic fraction was much higher for sample A. At this high iron fraction and small particle size, the iron atoms might not be effectively protected from oxidation, thus lowering the magnetic moment per iron atom of the particles. However, there is also the possibility that a composition limit is reached, whereby further increase in iron atomic composition beyond ˜52% will significantly increase the fraction of oxidized iron atoms in the particles regardless of whether the particles are captured in organic or aqueous solution. Further investigation on the magnetization of Au—Fe nanoparticles with iron compositions above 52% up to 70% needs to be done to determine optimum Au/Fe ratio for maximum particle magnetization.

TABLE 2
Magnetic and physical properties of Au—Fe nanoparticles
captured using mixed thiol-amine surfactants in mesitylene.

| | | | | | | | | | | Saturation | |
|---|---|---|---|---|---|---|---|---|---|---|---|
| | Particle Molar | | | | | Coercivity | | Remanence | | Magnetization | |
| Au—Fe | Composition | | Sample | Size | Average | Hc (Oe) | | Mr (emu/g) | | Ms (emu/g) | |
| Samples | Au | Fe | Weight (mg) | Range | Particle Size | 100K | 293K | 100K | 293K | 100K | 293K |
| 1 | 0.48 | 0.52 | 8.5 | 4-50 nm | 10 nm | 50 | 20 | 2.30 | 0.90 | 25.5 | 22.5 |
| 2 | 0.54 | 0.46 | 17.0 | 4-60 nm | 11 nm | 25 | 20 | 0.44 | 0.23 | 11.0 | 7.8 |
| 3 | 0.56 | 0.44 | 2.0 | 4-50 nm | 7 nm | 112 | 72 | 0.86 | 0.57 | 9.5 | 8.1 |
| 4 | 0.64 | 0.36 | 12.0 | 3-50 nm | 6 nm | 34 | 10 | 0.14 | 0.04 | 2.4 | 2.1 |

TABLE 3
Properties of Au—Fe nanoparticles captured using sodium citrate in water.

| | | | | | | | | | | Saturation | |
|---|---|---|---|---|---|---|---|---|---|---|---|
| | Particle Molar | | Sample | | Average | Coercivity, | | Remanence, | | Magnetization | |
| Au—Fe | Composition | | weight | Size | Particle | Hc (Oe) | | Mr (emu/g) | | Ms (emu/g) | |
| Samples | Au | Fe | (mg) | Range | Size | 100K | 293K | 100K | 293K | 100K | 293K |
| A | 0.44 | 0.56 | 2.1 | 4-20 nm | 6 nm | 35 | 7 | 6.20 | 0.68 | 9.7 | 8.2 |
| B | 0.56 | 0.44 | 2.1 | 3-35 nm | 5 nm | 45 | 30 | 6.10 | 2.64 | 10.0 | 8.2 |

Based on the long-term magnetic stability of the DACS nanoparticles, the iron atoms in the particles appear to be successfully protected from oxidation. However, the water captured Au—Fe nanoparticles have a lower saturation magnetization than the organic captured Au—Fe nanoparticles, and the magnetic moment per iron atom within the nanoparticles is much lower than the magnetic moment per iron atom in bulk iron, which is 2.2 μ_{B}/Fe atom, or in dilute Fe/Au bulk alloys, which is 2.6 μ_{B}/Fe atom. The iron atoms on the surface of the particles are most probably oxidized to α-Fe₂O₃ (haematite) and this may be the reason that the magnetic moment per iron atom in the particles is less than expected. There is also the possibility that some or all of the iron in the particles could be partially oxidized to a metastable magnetic state (Fe₃O₄ and γ-Fe₂O₃). Further investigation on the iron oxidation state, particle spin domains and the electron coupling between gold and iron needs to be done to better understand the magnetic behavior of these Au—Fe nanoparticles.

B: Variation in the Properties of DACS Au—Fe Nanoparticles

Tables 4 and 5 compare the properties of the Au—Fe nanoparticles captured by a permanent magnet (0.3 T) for magnetic measurements to the properties of the Au—Fe nanoparticles that remained in the solution, i.e. were not drawn out of the solution by the magnet. The nanoparticles not magnetically captured usually constitute about 10-20% of the total nanoparticle sample.

AA analysis of the Au—Fe nanoparticles not separated by the magnet shows that these nanoparticles have a lower gold content than the nanoparticles separated from solution by the magnet. Therefore, DACS Au—Fe nanoparticles do exhibit a composition variation from one particle to another. Surprisingly, the nanoparticles that are not separated by the magnet are richer in iron than those that are separated. It is speculated that these Au—Fe nanoparticles with lower gold content have low magnetic moments or simply are not magnetic due to a higher iron content on the particle surface or phase segregation within the particle to gold-rich and iron-rich regimes. Either case would expose more of the iron atoms to oxidation. Although Au—Fe particles that are very rich in iron have a tendency to form heterogeneous particles with an iron oxide layer surrounding a gold core, such structure was not obvious in the TEM micrographs of the Au—Fe nanoparticles not separated by the magnet. However, two-phase structures such as a dark hemisphere attaching to a lighter hemisphere were at times seen. When the gold and iron atoms phase segregate to form iron-rich or gold-rich phases, the iron atoms are most likely to be oxidized and lose their magnetic characteristics.

In addition to being richer in their iron content, the organic captured Au—Fe nanoparticles not separated by the magnet are generally smaller in size than those that are separated by the magnet. This is to be expected as gold atoms are known to have greater affinity towards each other than iron atoms do. Thus, particles with a higher fraction of gold atoms on their surface will tend to coalesce to produce larger particles. Also, since the fraction of protected core iron atoms decreases with decreasing particle size, the magnetic moment of a small particle is likely to be significantly lower than that of a bigger particle with the same composition. Therefore, nanoparticles with small diameters and high iron content are most likely to have low specific magnetic moments. Unlike the organic captured Au—Fe nanoparticles, the water captured Au—Fe nanoparticles not drawn to the magnet have the same average particle size as the ones drawn to the magnet. Since the water captured nanoparticles are generally very small (average diameter below 8 nm), the magnetic properties of these nanoparticles are largely dependent on the particle composition and how the gold and iron atoms are distributed within a particle.

TABLE 4
Physical properties of Au—Fe nanoparticles captured in organic solution with
respect to whether the particles are captured by a permanent magnet or not.

| | Captured | | | | Not Captured | | | |
|---|---|---|---|---|---|---|---|---|
| Au—Fe | Particle Composition | | Particle | Average | Particle Composition | | Particle | Average |
| Samples | Au | Fe | Size | Size | Au | Fe | Size | Size |
| 1 | 0.48 | 0.52 | 4-50 nm | 10 nm | 0.41 | 0.59 | 4-12 nm | 8 nm |
| 3 | 0.56 | 0.44 | 4-50 nm | 7 nm | 0.44 | 0.56 | 3-24 nm | 5 nm |
| 4 | 0.64 | 0.36 | 3-50 nm | 6 nm | 0.61 | 0.39 | 3-30 nm | 5.5 nm |

TABLE 5
Particle composition of Au—Fe nanoparticles originally
captured in citrate/water solution with respect to whether
the particles are captured by a permanent magnet or not.

| | | Particle Composition (mol/mol) | |
|---|---|---|---|
| | | Au | Fe |
| Au—Fe Sample 1: | In Bulk Solution | 0.36 | 0.64 |
| | Captured | 0.44 | 0.56 |
| | Not Captured | 0.34 | 0.66 |
| Au—Fe Sample 2: | In Bulk Solution | 0.46 | 0.54 |
| | Captured | 0.56 | 0.44 |
| | Not Captured | 0.32 | 0.68 |

### Example VII

### Preparation of Fe/Au Nanoparticles for Bulk Magnetization Measurements

The Fe(50)/Au(50) nanoparticles whose magnetization curves are shown in FIG. 6 were prepared using the Distributed Arc Cluster Source (DACS) shown in FIG. 1. Gold and iron metals with 99.9% purity were purchased from Alfa Aesar (Ward Hill, Massachusetts). The DACS has a positively biased carbon rod which supports the tungsten feed crucible, and a negatively biased tungsten rod of 0.06 inches diameter which provides a sharp point for effective plasma arc generation. During the operation, argon gas is continuously fed from the bottom of the DACS column to serve as a carrier gas for the metal vapor. Argon also serves as a precursor for arc generation.

The positively charged feed crucible was raised until the metal charge in the crucible comes in contact with the negatively charged tungsten rod. The electrical spark that results ionized the argon gas and a plasma arc formed between the tungsten rod and the metal charge in the crucible. The crucible is then lowered a fixed distance to establish a predetermined arc voltage drop. The plasma arc has a temperature as high as 4000 K and provides the heat necessary to evaporate the metal charge. After arc initiation, the arc was maintained primarily by the ionized metal vapor from the feed rather than argon. The temperature outside of the plasma arc is much lower than the temperature in the arc itself. Gas phase nanoparticles were formed when the metal vapor is swept upstream by the argon gas. Helium quench gas at room temperature was mixed with the flow from the arc region and this further cooled the nanoparticles.

The aerosol stream from the DACS was bubbled into a 130 ml capacity capture cell made of Pyrex glass (FIG. 2). The capture cell is a 19″ long cylinder with a 1.5″ diameter and contains 6 Teflon baffles, which provide good liquid-gas contact The capture cell contained a solution of 4.2 mmol dodecanethiol, 0.27 mmol dodecylamine, and 0.17 mmol didecylamine in 120 ml of mesitylene. All chemicals were purchased from Aldrich. The mesitylene was 97% pure and the surfactant molecules were all 98% pure.

After a run of approximately 15 minutes the DACS was shut down and the solution in the capture cell now containing Fe/Au nanoparticles in suspension was allowed to settle for an hour and then was transferred into a separatory flask. The solution was allowed to flow through a Tygon tube nominally 0.25″ in diameter past a 0.3 T permanent magnet, which caused the entrained Fe/Au nanoparticles to collect on the wall of the tube at the location of the magnet. This bulk sample was air dried and weighed. It was then mixed with epoxy and placed in a plastic straw for insertion into a Quantum Design MPMS SQUID Magnetometer for the magnetization measurements. The magnetization curves were obtained in the laboratory of Professor Sarah Majetich at Carnegie Mellon University.

Separate measurements on this sample yielded an average particle size of 10 nm and a composition of Fe(50)/Au(50).

### Example VIII

### Detection of DNA Using Functionalized Fe/Au Nanoparticle

A: Materials and Methods

Reagents. HAuCl₄.3H₂O was obtained from Aldrich Chemical Co. All other chemicals such as NaCl, KCl, N₃C₆H₅O₇, NaH₂PO₄, and Na₂HPO₄ were obtained from Mallinckrodt Chemical Company (Philipsburg, N.J.). Colloidal gold nanoparticles with an average diameter of 13 nm were prepared according to the literature by reduction of HAuCl₄ with Na₃C₆H₅O₇ aqueous solution. 5′ alkyl and 3′ alkyl thiolated (HO—(CH₂)₆S—S(CH)₆-modified) single-stranded oligonucleotides were obtained from Integrated DNA Technologies (Iowa City, Iowa). The sequence of the oligonucleotides, after cleavage, was as follows: 5′ HS-(CH₂)₆-GTC AGT CCG TCA GTC-3′ (DNA-1) (SEQ ID NO: 1) and 5′-ATG CTC AAC TCT CCG-(CH₂)₆—SH 3′ (DNA-2) (SEQ ID NO:2). Dithiothreitol (DTT) was procured from Sigma Chemical Co. Disulfide bonds on the single stranded oligonucleotides were cleaved with 100 mM DTT in 0.17 M Na₂HPO₄/NaH₂PO₄ solution at pH=8.0 and desalted with NAP-5 columns, purchased from Pharmacia Biotech. The water used in this study was treated with a Milli-Q gradient water purification system with a photo-oxidation source (Millipore, Bedford, Mass.).

B: Preparation of Au Particles.

All glassware used in this study was cleaned in aqua regia (3:1 v/v with HCl:HNO₃), rinsed thoroughly in Milli-Q water (Millipore), and ovendried prior to use. An aqueous solution of HAuCl₄ (1 mM, 200 mL) was brought to a reflux while stirring, and then 17.5 mL of a 38.8 mM Na3C₆H₅O₇ solution was added quickly. After the color change, the solution was refluxed for an additional 15 minutes, allowed to cool to room temperature, and subsequently filtered through a 0.8 μm Gelman syringe filter. The gold colloidal particles were characterized by UV-Vis spectrometry and transmission electron microscopy (TEM). A typical solution of 13 nm diameter gold particles exhibited a characteristic surface plasmon band centered at 520 nm. The average size and size distribution for the colloidal particles were determined with TEM image.

C: Preparation of Fe/Au Nanoparticles.

The Fe/Au nanoparticles are prepared by an aerosol process using the Distributed Arc Cluster Source (DACS) shown in FIG. 1. Gold and iron metals with 99.9% purity were purchased from Alfa Aesar (Ward Hill, Mass.). The DACS has a positively biased carbon rod which supports the tungsten feed crucible, and a negatively biased tungsten rod of 0.06 inches diameter which provides a sharp point for effective plasma arc generation. During the operation, argon gas is continuously fed from the bottom of the DACS column to serve as a carrier gas for the metal vapor. Argon also serves as a precursor for arc generation.

The positively charged feed crucible is raised until the metal charge in the crucible comes in contact with the negatively charged tungsten rod. The electrical spark that results ionizes the argon gas and a plasma arc forms between the tungsten rod and the metal charge in the crucible. The crucible is then lowered a fixed distance to establish a predetermined arc voltage drop. The plasma arc has a temperature as high as 4000 K and provides the heat necessary to evaporate the metal charge. After arc initiation, the arc is maintained primarily by the ionized metal vapor from the feed rather than argon. The temperature outside of the plasma arc is much lower than the temperature in the arc itself. Gas phase nanoparticles are formed when the metal vapor is swept upstream by the argon gas. A quench gas (helium or nitrogen) at room temperature is mixed with the flow from the arc region and this further cools the nanoparticles.

The Fe/Au particles are collected from the gas phase in the capture cell (FIG. 2). The particles in these experiments were captured in a dilute citrate solution.

The size of the particles formed is dependent on the evaporation rate and how fast the metal vapor is removed form the arc region. These conditions can be controlled by controlling the arc power, by adjusting the distance between the tungsten electrode and the metal charge in the crucible, and by adjusting the flow rates of the carrier and quench gases.

D: Preparation of DNA Conjugated Au Nanoparticle.

The 5′ disulfide bond of the 5′ HO—(CH₂)₆S—S(CH₂)₆-modified oligonucleotides was cleaved prior to surface modification. The DNA-modified gold nanoparticle solution was prepared as following. For each oligonucleotide, a solution of Au nanoparticles (˜17nM, 1 mL) was combined with 1:1 (w/v) of 3-6 μM DNA. After standing for 24 hours at room temperature, the solution were diluted to 0.1 M NaCl, 10 mM Na₂HPO₄/NaH₂PO₄ (pH 7.0) and allowed to stand for 40 hours, followed by centrifugation at 12800 rpm for 25 minutes to remove excess DNA. Following removal of the supernatant, the DNA modified gold nanoparticles were resuspended in 0.5 M NaCl, and 10 mM Na₂HPO₄/NaH₂PO₄, which is suitable for DNA hybridization.

E: Preparation of DNA Conjugated Fe/Au Nanoparticles.

The DNA conjugation to Fe/Au nanoparticles was performed using the procedure described above for the Au particles. 5′-ATG CTC AAC TCT CCG-(CH₂)₆—SH 3′ (SEQ ID NO:2) was conjugated to the Fe/Au nanoparticles synthesized using DACS in a 1:1 (w/v) solution of 3-6 μM DNA. The average size of the particles and the size distribution was determined with TEM measurement.

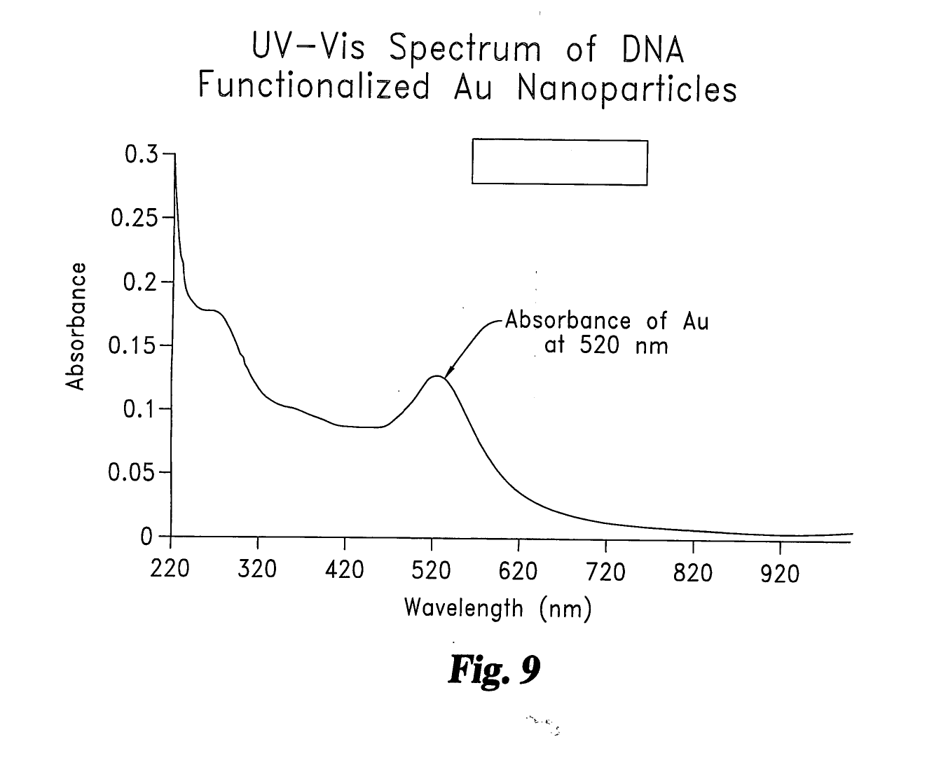

F: Optical Signature of DNA Functionalized Particles Optical properties of the functionalized particles were examined by UV-Vis spectrometry. FIG. 9 shows the UV-Vis absorbance spectra for 20 nm diameter Au particles functionalized with DNA-B. The absorbance peak in the 500-600 nm region is due to an inelastic resonance, which is characteristic of Au and Ag particles and which results in a larger than usual optical scattering cross-section for these metal nanoparticles.

FIG. 10 shows the UV-Vis absorbance spectra for 10 nm diameter Fe/Au particles taken both before and after functionalization with DNA-A. There is no significant optical signature with Fe/Au solution, however, there is small shoulder near the 530 nm mainly due to the portion of Au atoms (series 1). This characterization didn't change after DNA modification, indicative there is no significant particle aggregation (series 2). The lack of an absorbance peak in the case of the Fe/Au particles indicates the lack of a strong resonance absorption as compared to the Au nanoparticles. This results in a lower optical scattering cross-section for the Fe/Au particles and allows optical discrimination between Au and Fe/Au particles.

G: Binding of DNA/Au Nanoparticles Particles to Target DNA

Colloidal 13 nm diameter Au particles form a dark red suspension in H₂O, and like thin film Au substrates, they are easily modified with oligonucleotides that are functionalized with alkanethiols at either or both of their 5′ and 3′ ends. These oligonucleotide modified Au nanoparticles exhibited high stability in solution containing elevated salt concentrations and elevated temperature, an environment that is incompatible with unmodified particles.

Two species of functionalized Au particles were created: one using the 15-mer 5′ HS—(CH)₆-GTC AGT CCG TCA GTC-3′ (DNA-1) (SEQ ID NO:1) and one using the 15-mer 5′-ATG CTC AAC TCT CCG-(CH)₆—SH 3′ (SEQ ID NO:2). Portions of each of these two colloidal DNA conjugated Au nanoparticle solutions were combined, and because of the non-complementary nature of the oligonucleotides (SEQ ID NOs: 1 and 2) attached to the particles, no reaction took place, i.e., the UV-Vis spectrum didn't change.

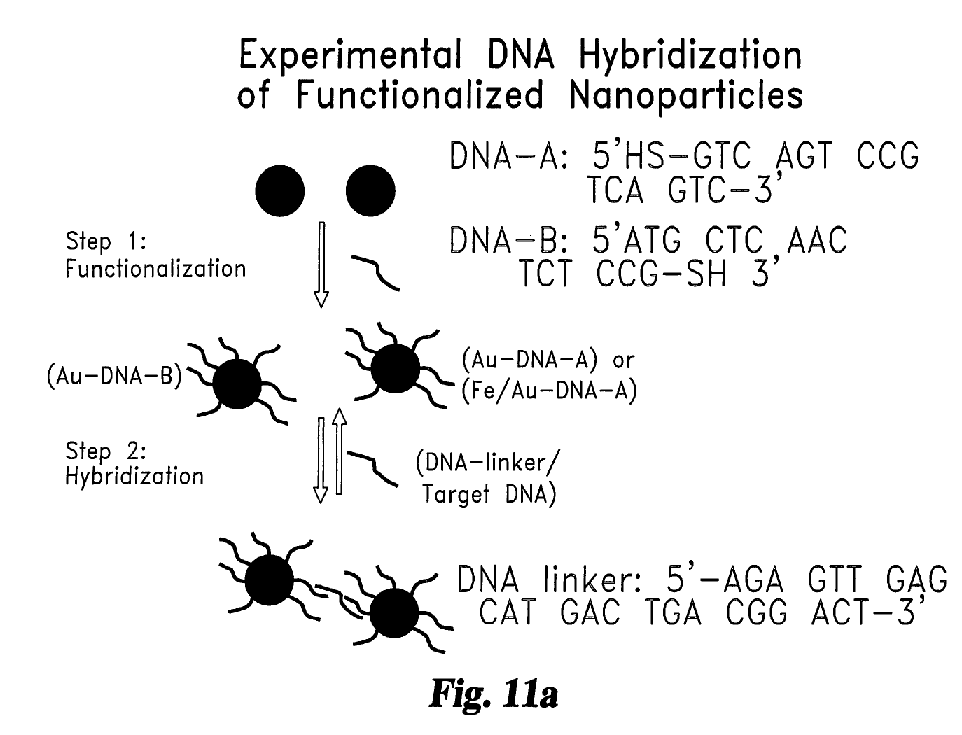

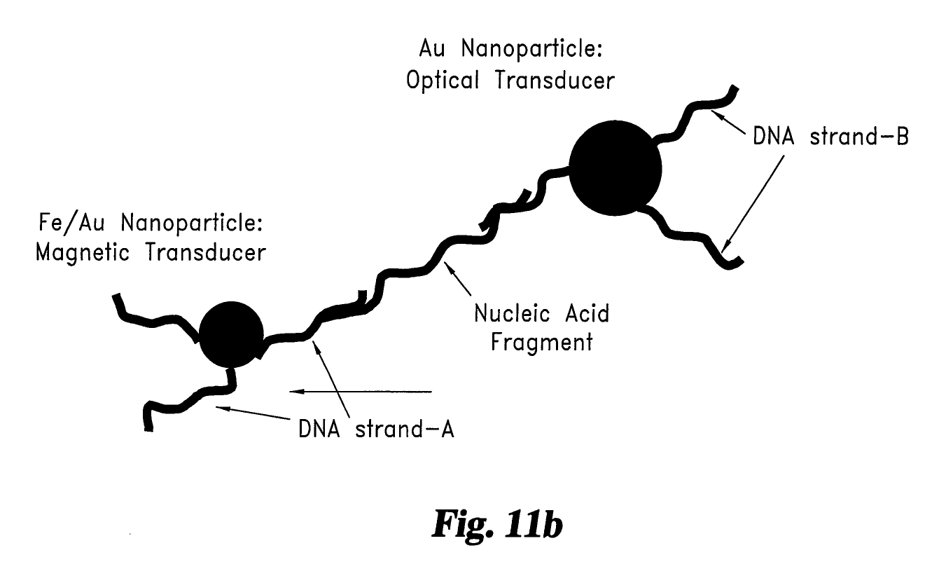

The solution containing the two species of DNA conjugated Au particles was combined with a solution containing 2 nmol of a DNA linker (substrate) consisting of the 24-mer 5′ AGA GTT GAG CAT GAC TGA CGG ACT-3′ (SEQ ID NO:3). This linker hybridizes with both DNA sequences attached to the Au nanoparticles, but at different 12 base pair regions. FIG. 11a shows the experimental design. Significantly, an immediate color change from red to purple was observed, and a precipitation reaction ensued. Over the course of several hours, the solution became clear, and a pinkish gray precipitate settled to the bottom of the reaction cuvette. This occurred because DNA linker molecules hybridized with the many complementary oligonucleotides anchored to the Au nanoparticles, thereby cross linking them (to yield what we term Au:DNA:Au complexes), which resulted in the formation of dark precipitation. When the cuvette containing the precipitate was heated to the 60 degrees, the red color of the solution returned, indicative of the denaturation (melting) of the hybridization complexes and hence the unlinking of the nanoparticles. However, when the solution was allowed to stand at room temperature after heating, the color changes and precipitation process again took place.

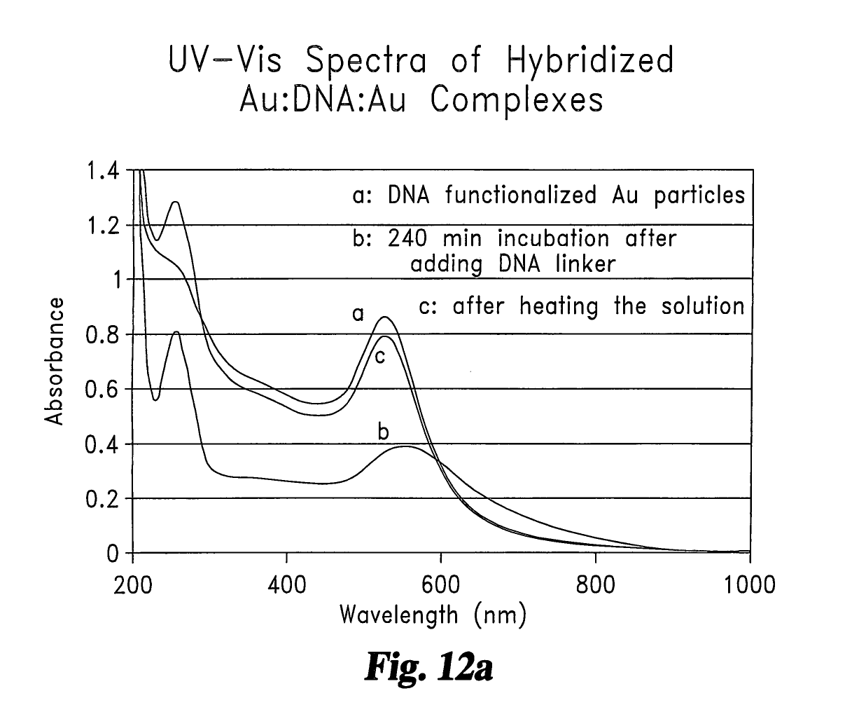

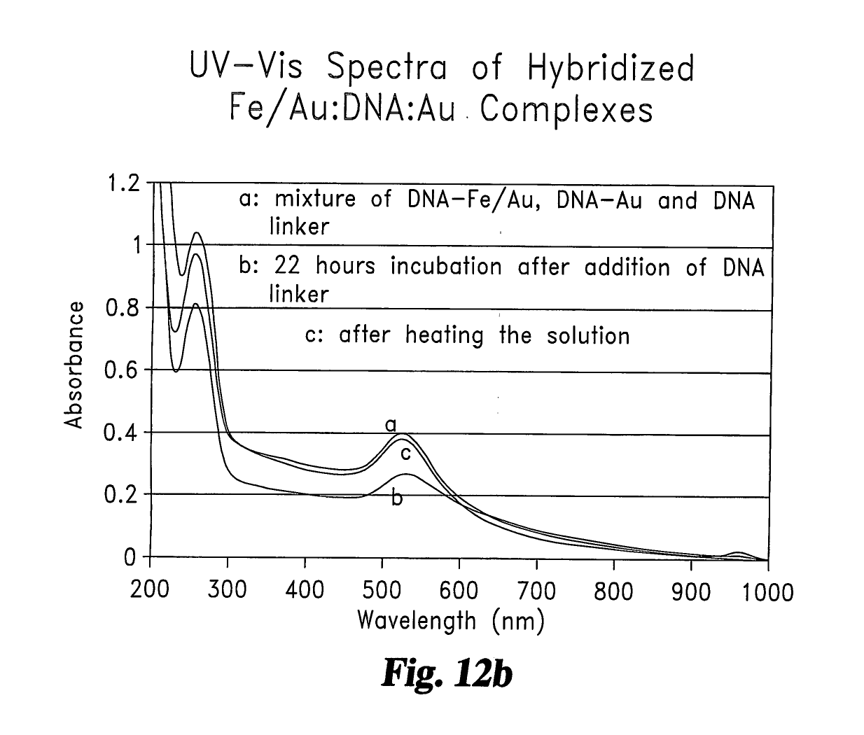

These optical changes were monitored by UV-Vis spectrometer in FIG. 12a The spectral changes associated with the nanoparticle assembly process (spectrum b) include a broadening and red shift in the plasmon resonance band, centered near 520 nm for the unlinked nanoparticles, and a concomitant decrease in the absorbance at 260 nm. The plasmon band shift is attributed to the electromagnetic interactions of the particles as the interparticle distance decreases with hybridization. The lowering and red shifting of the absorbance peak in the 500-600 nm region is due to the formation of Au particle:DNA linker:Au particle complexes and their gradual precipitation from the solution (Nature 382, 607 (1996)). The temperature at which these spectral changes occurred for the nanoparticle assembly were correlated with the DNA hybridization process. TEM showed the Au nanoparticle aggregated due to the DNA hybridization.

H: Binding of DNA/Au/Fe and DNA/Au Nanoparticles Particles to Target DNA

A DNA targeting experiment was conducted using functionalized Au/Fe particles (derivatized with the 15-mer 5′-ATG CTC AAC TCT CCG-(CH₂)₆—SH 3′; SEQ ID NO:2) and functionalized Au particles (derivatized with the 15-mer 5′ HS—(CH₂)₆-GTC AGT CCG TCA GTC-3′; SEQ ID NO:1). Portions of each of these two colloidal DNA conjugated Au nanoparticle solutions were combined to allow for the DNA hybridization reaction. Again, because of the non-complementary nature of the oligonucleotide attached to the particles, no reaction took place. Since the Fe/Au solution does not contain strong optical signature, only the Au solution signature was observed, as strong peak at 525 nm. After DNA linker substrate was added, no immediate color changes were observed. However, there was some red shift due to the DNA hybridized Fe/Au and Au nanoparticles (what we term Fe/Au:DNA:Au complexes). FIG. 11b shows the smallest such complex formed in the hybridization reaction.

These optical changes were monitored by UV-Vis spectrometer in FIG. 12b. After 22 hours, the peak shifted to 535 nm and the intensity was decreased as we observed DNA hybridized Au nanoparticles. The lowering and red shifting of the absorbance peak in the 500-600 nm region is visibly less than is the case for the experiment illustrated in FIG. 11a. The lowering of the peak is due to the formation of Fe/Au particle:DNA linker:Au particle complexes and their gradual precipitation from the solution. The absence of a decided red shift is due to the lower dipole-dipole coupling between Fe/Au and Au particles in the present complexes as compared to the dipole-dipole coupling between Au particles in the complexes that form in the experiment illustrated in FIG. 11a. The degree of the red shift was not significant compared to the DNA hybridized Au nanoparticles. This is attributed to the fact that the optical change is mainly due to the extent of the particle aggregation. The DNA hybridized Fe/Au and Au nanoparticles were heated to 60 degrees, the denaturation (melting) temperature of the DNA linker, and red color returned due to the denaturation of the DNA and the resulting monodispersed Fe/Au and Au nanoparticles. This is indicative of 1) there is indeed DNA attached to the Fe/Au nanoparticles, and 2) all the DNA attached to the Fe/Au particles were functional. The functionalized Fe/Au particles behaved like functionalized Au particles in that they bound to the DNA fragment and produced similar complexes.

### Example IX

### Detection of Virus Using Antibody-Functionalized Au Nanoparticle

Au nanoparticles (10 nm and 20 nm in diameter) and Fe/Au nanoparticles (10 nm in diameter) were prepared as described in the preceding example. An anti-phage M13 antibody, anti-PVIII, was used to conjugate the nanoparticles. The pH of the Au nanoparticle solution was adjusted to between pH 8 and pH 9. Anti-PVIII antibody (15 mL of a 1 mg/mL solution) was added to 1 mL of 10 nm diameter Au nanoparticle solution. To conjugate the 20 nm diameter Au nanoparticles, twice the amount of anti-PVIII antibody was used. Conjugated Fe/Au particles were made in a similar fashion. The final solutions additionally contained about 1%, by weight, bovine serum albumin (BSA) for stabilization. The solutions were centrifuged to remove excess antibody, and the conjugated Au nanoparticles and Fe/Au nanoparticles were resuspended in 12 mM phosphate buffered saline.

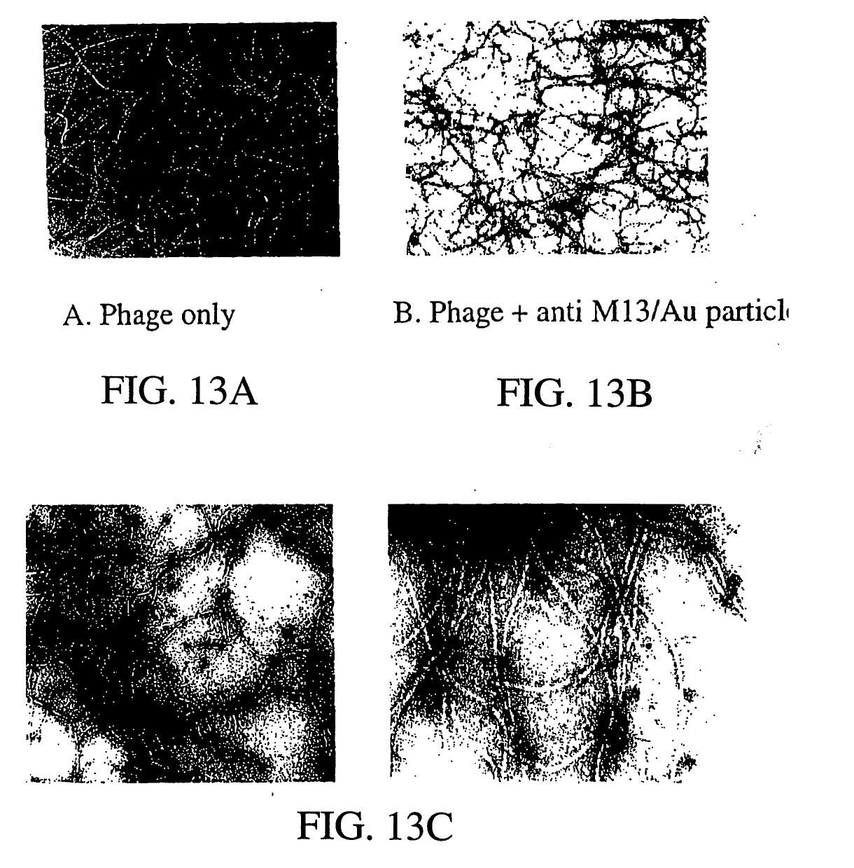

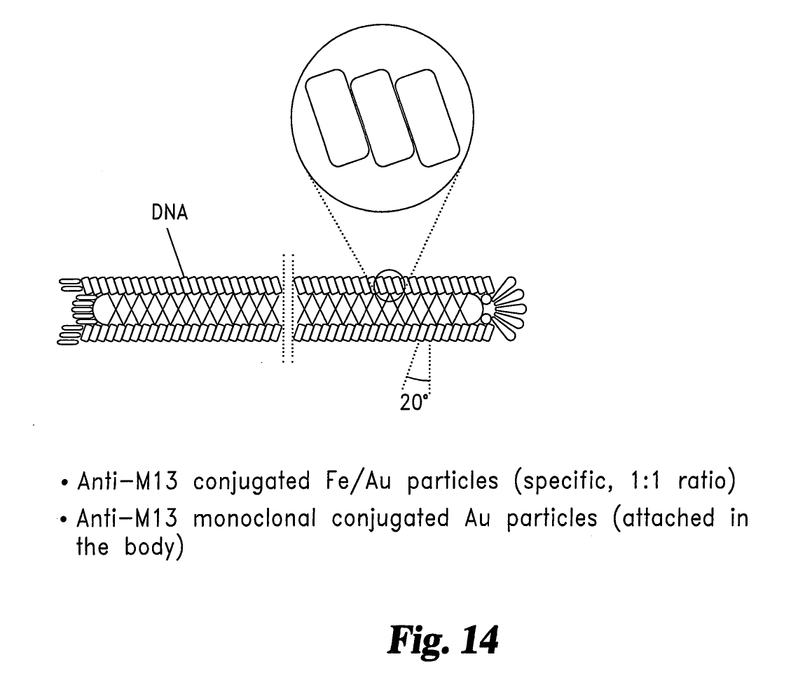

Antibody-conjugated Au and Fe/Au nanoparticles were contacted to phage M13. As shown in the TEM images set forth in FIG. 13, the antibody-conjugated Au nanoparticles bound specifically to phage M13. FIG. 14 shows an experimental design for detecting M13 phage using anti-M13 conjugated Fe/Au particles and/or anti-M13 monoclonal conjugated Au particles.

A magnet was used to pull the Fe/Au-bound viruses out of solution, the bound complexes were resuspended, and the solution was observed with an optical microscope. Elongated shaped objects were observed that appear to be viruses decorated with Au particles. The viruses were observable because they are 1,000 nm×8 nm in size, and the Au nanoparticles that bind to them scatters the light very strongly.

### Example X

### Selective Capture of Fe/Au Nanoparticles from a Solution Containing Au Nanoparticles

Bound complexes between magnetic particles and biological species can be manipulated in solution by the application of an external magnetic field. In this way they can be separated from non-magnetic species and concentrated. What differentiates the complexes of the invention from previous art that utilizes micron-scale magnetic particles is the large magnetic susceptibilities per volume of the Fe/Au particles. Thus, it is possible with application of a modest magnetic field to manipulate Fe/Au:target complexes in which the Fe/Au particles are only a few nanometers in diameter. When a micron-scale magnetic particle is collected there is no way of determining whether it has a biological target attached unless the biological target is large enough so that it is distinguishable from the magnetic particle. In the case of a nano-scale magnetic particle, however, determination of whether it has a target species attached is often possible. One method for obtaining such a determination is to introduce a nano-scale optical marker that is only present when the biological target is present. Detecting the optical marker associated with a magnetic nanoparticle is then tantamount to determining the presence of the biological target.

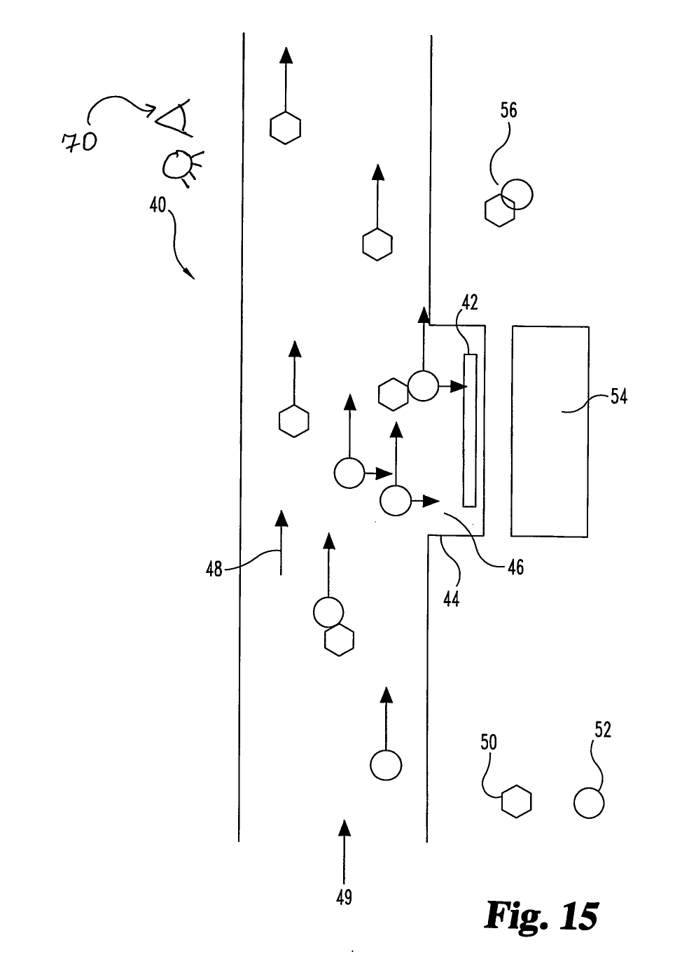

Both Au and Fe/Au nanoparticles can be functionalized so that they selectively bind to biological targets. It is also possible to differentiate between Au and Fe/Au nanoparticles of the same size either by the difference in their electron scattering density using transmission electron microscopy or by the difference in their optical absorption cross sections using phase contrast imaging. Thus, combining functionalized Fe/Au nanoparticles to act as magnetic carriers and functionalized Au nanoparticles to act as optical markers is an attractive approach to selective, sensitive detection of biological targets. The essence of the scheme is to introduce both nanoparticle reagents into the solution believed to contain the target species and to allow Fe/Au particle:target species:Au particle complexes to form. If perfect separation of magnetic species and non-magnetic species can be achieved in a device such as shown schematically in FIG. 15, counting the number of Au particles collected is then equivalent to counting the number of target species collected.

Clean separation of magnetic and non-magnetic nanoparticles based on their relative mobility in solution is difficult due to the large diffusion mobility of these ultra-small species. Although it is relatively easy to harvest magnetic particles by flowing a solution containing the particles past a fixed magnet, there is always a substantial population of non-magnetic particles that is also collected due to the random diffusive motion of these species in the solution. Thus, when a substrate is placed in a flowing stream there is always a background signal of non-magnetic nanoparticles collected along with the magnetic particles. A scheme that minimizes this background and thereby increases the sensitivity of detection is presented here. It will be understood that the solution need not flow past or through the device, but may be a non-flowing sample that has been collected from another source.

By placing a collection substrate 42 (in this case, a TEM grid) in a recessed cavity 44 as shown schematically in FIG. 15, it is possible to maintain a thin stagnant liquid layer 46 between the substrate 42 and the liquid 48 flowing in a channel 49. This stagnant liquid layer 46 serves as a diffusion barrier that can varied merely by adjusting the depth of the cavity 44. The flow in the channel 49 can be adjusted so that less than one 20 nm diameter Au particle 50 per about 10¹⁶ in the solution flowing past the capture substrate is deposited on the substrate. Using this configuration it was possible to achieve almost perfect separation between Fe/Au 52 and Au nanoparticles 50 in aqueous solution.

The capture cell consisted of a 1 mm high by 8 mm wide channel machined in a Teflon block. A copper TEM grid coated with a thin carbon film was placed in a circular cavity 5 mm in diameter and 0.1 mm deep that was centered over a 1.2 cm diameter, 0.3 T magnet 54. Two solutions were prepared. One consisted of 20 nm diameter Au particles suspended in a 1.0 millmolar solution of sodium citrate and DI water. The second consisted of an equimolar mixture of Fe(50)/Au(50) particles having a mean diameter of 30 nm and 20 nm diameter Au particles suspended in a 1.0 millimolar solution of sodium citrate and DI water. The approximate concentration of nanoparticles in each solution was 5×10¹⁰ particles/ml or ˜10⁻¹⁵ molar.

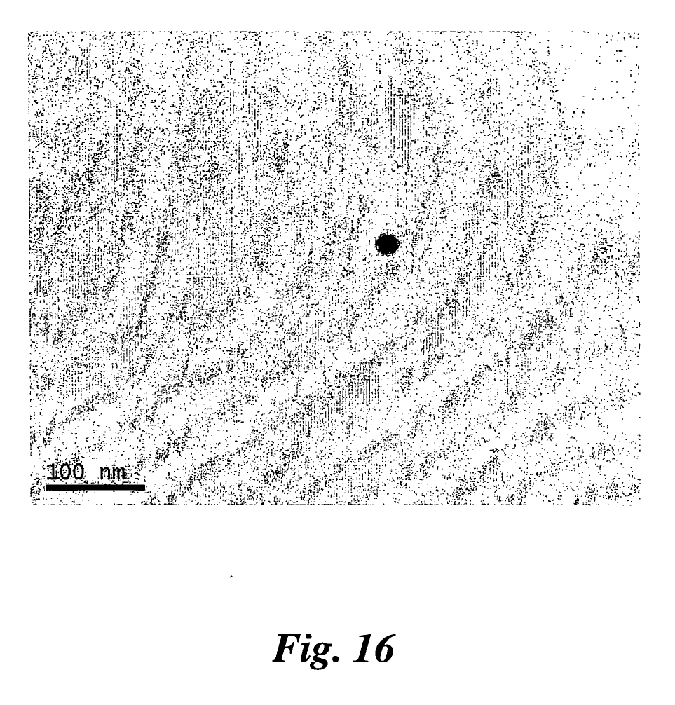

The effectiveness of the stagnant liquid layer as a diffusion barrier was tested by flowing approximately 50 ml of the first solution at a rate of 10 ml/min through the capture cell. Inspection of the TEM substrate in a JEOL 2000 FX transmission electron microscope revealed an essentially bare substrate. The TEM image in FIG. 16 shows one Au particle, but it was so difficult to find Au particles that it was impossible to compute an areal density.

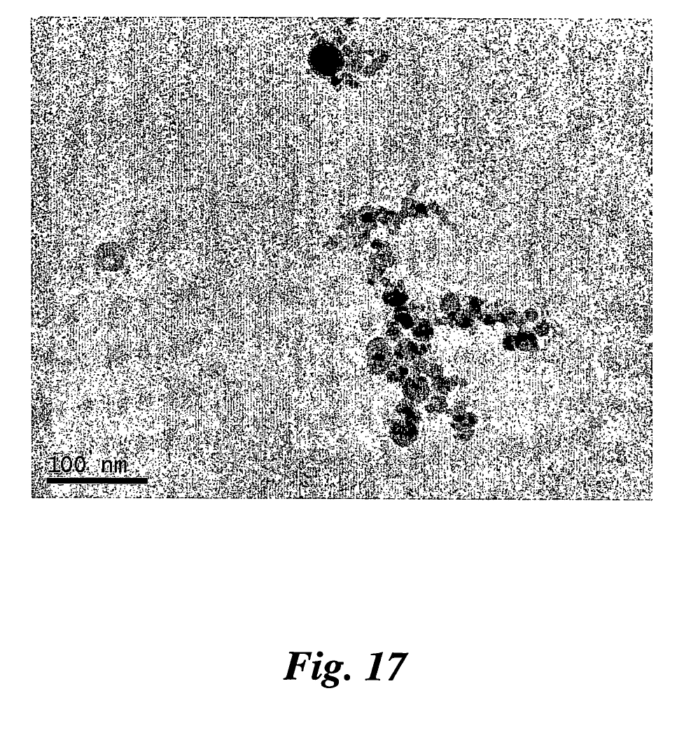

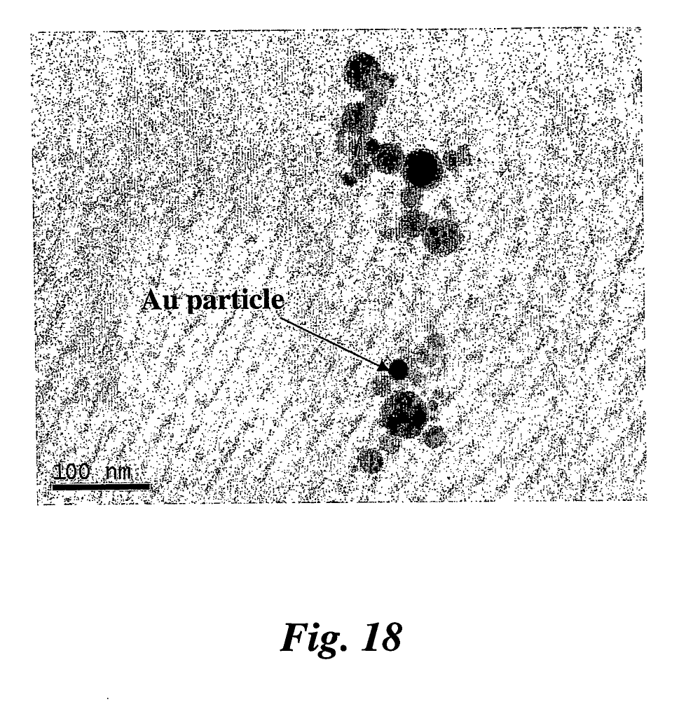

Next 50 ml of the second solution was passed through the cell at a rate of 10 ml/min. Inspection of the TEM substrate now revealed a large concentration of Fe/Au nanoparticles 52 that were collected due to the magnetic field. A pair of typical TEM images are shown in FIGS. 17 and 18. There are no Au nanoparticles visible in micrograph of FIG. 17 or in other representative micrographs taken from the same TEM substrate. The size distribution of the Fe/Au nanoparticles is quite large as no attempt was made to size select them, but it is possible to determine that no Au particles are present from the intensity of the TEM images. It should also be possible to resolve an Au particle in the presence of a lot of Fe/Au nanoparticles by the difference in their optical images. The approximate areal density of Fe/Au particles in was measured to be ˜1×10¹⁰ particles/cm². Extensive searching revealed an occasional Au particle such as is shown in FIG. 16, but again, the number of Au particles on the substrate was two low to count.

Referring again to FIG. 15, these experiments serve to show the feasibility of selective collection of Fe/Au nanoparticle:biological target:Au nanoparticle complexes 56 in a cell in which negligible collection of free Au particles 50 takes place. Each complex 56 that was captured and deposited on the substrate 54 because of the presence of a magnetic Fe/Au particle 52 or particles in the complex 56 would contain one or more optically detectable Au particles. The absolute collection efficiency of the model cell for the Fe/Au particles 52 in the experiments described is low, however, this efficiency can be easily increased by scaling down the depth of the flow channel while keeping the Reynolds number of the flow constant. The experiments also demonstrate the feasibility of detecting Au nanoparticles 50 in the presence of a much larger number of Fe/Au nanoparticles 52. Although this has only been demonstrated using TEM detection, it is expected that optical detection will also provide excellent discrimination and as optical detection is much cheaper than TEM, it is preferred.

The flow cell depicted schematically in FIG. 15 can also include an optional detector 70, which can be placed downstream of the magnet 54. This detector can be used to detect Au nanoparticles 50 in liquid 48 and can be used in addition to or in the alternative to any detection or measurement obtained on collection substrate 42. Detector 70 can be any detection device capable of detecting free Au nanoparticles in solution including optical detection methods described herein. Detecting the Au nanoparticles in solution can be used to determine the presence and concentration (or amount) of the target material in a sample. This can be particularly effective when a known amount, which would be a predicted excess amount, of the functionalized Fe/Au and Au particles are added to the sample suspected of containing the target material. The functionalized Fe/Au and Au particles will bind to essentially all of the target material to yield bound complexes. The magnet would remove these bound complexes leaving a residual amount of the functionalized Au nanoparticle in the sample. This residual amount of Au particles can be detected and quantified. The difference between the known starting amount of Au particles and the residual amount could be correlated to the amount of target material in the sample.

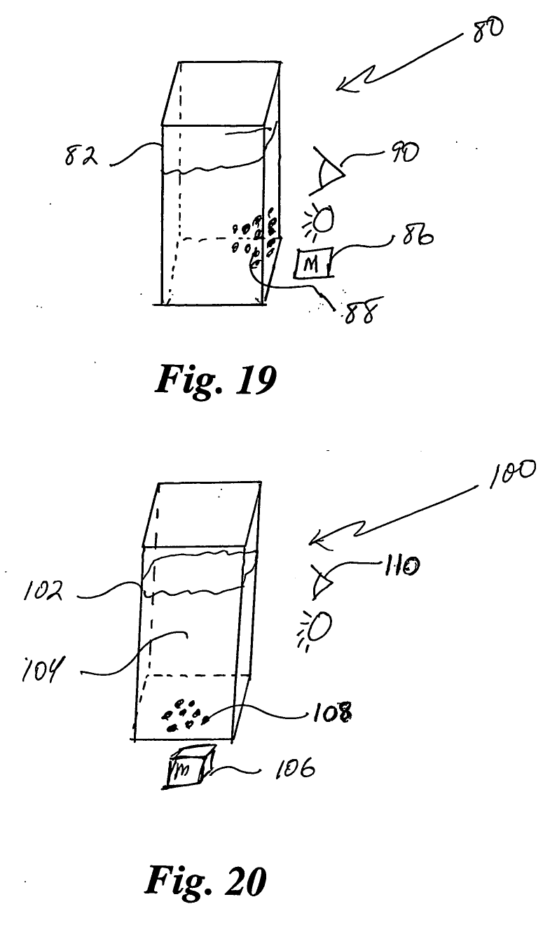

FIG. 19 illustrates another embodiment of a device for use in the present invention. Device 80 includes a container 82 such as test tube or cuvette that holds at least a portion of a sample fluid 84 including a target material. Sample fluid in this embodiment is a stagnant fluid. Functionalized Fe/Au and Au nanoparticles, which can be bound to each other or not bound together, are added to sample 84. A magnet 86 can be positioned proximate to a side wall of container 82 to attract the bound complex 88 that includes bound target material and both the functionalized Fe/Au nanoparticles and the functionalized Au nanoparticles. A detector 90 is positioned adjacent to magnet 86 to detect the Au nanoparticles in the bound complex.

FIG. 20 illustrates yet another embodiment of a device 100 for use in the present invention. Device 100, similar to device 80 includes a container 102 into which a sample 104 has been placed. Sample 104 contains or is suspected to contain a target material. Functionalized Fe/Au and Au nanoparticles, which can be bound to each other or not bound together, are added to sample 104. The Fe/Au particles bind to the target material to yield a bound complex 108. The Au nanoparticles are not included in the bound complex 108. A magnet 106 is positioned adjacent to one side of container 102 and attracts the bound complex 108, which are separated from the bulk sample 104. A detector 110 is positioned adjacent container 102 and spaced from magnet 106. Detector 110 can be used to detect that presence and amount of the Au nanoparticles in sample 104. The amount or concentration of the Au nanoparticles remaining or suspended in sampler 104 can be correlated to the amount or concentration of the target material in the original sample.

The detailed descriptions and examples included herein have been provided for clarity of understanding only. No unnecessary limitations are to be understood therefrom. The invention is not limited to the exact details shown and described; many variations will be apparent to one skilled in the art and are intended to be included within the invention defined by the claims. It is to be understood that the particular examples, materials, amounts, and procedures are to be interpreted broadly in accordance with the scope and spirit of the invention as set forth herein.

The complete disclosures of all patents, patent applications including provisional patent applications, and publications, and electronically available material (e.g., GenBank amino acid and nucleotide sequence submissions) cited herein are incorporated by reference.

1. A method of detecting a magnetic particle, said method comprising:
placing a first magnetic particle at a first location in a fluid medium;
applying a magnetic flux through a portion of the medium including the first location; and
observing movement of the magnetic particle in the fluid medium from the first location to a second location.

2. The method of claim 1 wherein the magnetic particle comprises a Fe/Au nanoparticle having at least one binding agent attached thereto.

3. The method of claim 1 wherein the magnetic particle comprises a Au nanoparticle different from the Fe/Au nanoparticle.

4. The method of claim 3 wherein the Au nanoparticle is attached to the at least one binding agent.

5. The method of claim 3 wherein the Fe/Au nanoparticle comprises a first binding agent and the Au nanoparticle comprises a second binding agent different from the first binding agent.

6. The method of claim 5 wherein the first binding agent comprises a first single stranded DNA fragment and the second binding agent comprises a second single stranded DNA fragment capable of hybridizing to at least a portion of the first DNA fragment.

7. The method of claim 5 wherein the first binding agent binds to the second binding agent.

8. The method of claim 7 wherein the target material displaces the second binding agent from the first binding agent.

9. The method of claim 2 wherein the magnetic particle comprises a bound magnetic transducer having a target material attached to the at least one binding agent.

10. The method of claim 9 wherein a Au nanoparticle is attached to the at least one binding agent.

11. The method of claim 9 wherein the bound magnetic transducer comprises a Au nanoparticle having at least one second binding agent different from the at least one first binding agent.

12. The method of claim 9 wherein the at least one binding agent comprises a first single stranded DNA fragment and the at least one second binding agent comprises a second single stranded DNA fragment capable of hybridizing to at least a portion of the first DNA fragment.

13. The method of claim 9 wherein the target material comprises a DNA fragment or an antigen.

14. The method of claim 9 wherein the target material is selected from group consisting of: proteins, peptides, carbohydrates polysaccharides, glycoproteins, lipids, hormones, receptors, antigens, allergens, antibodies, substrates, metabolites, cofactors, inhibitors, drugs, pharmaceuticals, nutrients, toxins, poisons, explosives, pesticides, chemical warfare agents, biohazardous agents, vitamins, heterocyclic aromatic compounds, carcinogens, mutagens, narcotics, amphetamines, barbiturates, hallucinogens, waste products.

15. The method of claim 9 wherein the bound magnetic transducer comprises both a Fe/Au nanoparticle and a Au nanoparticle.

16. The method of claim 9 wherein the bound magnetic transducer comprises a plurality of Fe/Au nanoparticles and a plurality of Au nanoparticles.

17. The method of claim 1 wherein the medium is an aqueous medium.

18. The method of claim 1 wherein the medium comprises agarose.

19. The method of claim 1 wherein said observing comprises optically detecting the magnetic particle.

20. The method of claim 1 wherein said optically detecting comprises detecting electron scattering density using transmission electron microscopy techniques.

21. The method of claim 1 wherein said optically detecting comprises detecting a fluorescent, radioactive, chemiluminescent, electrochemiciluminescent, or enzymatically labeled agent.

22. The method of claim 1 comprising a second magnetic particle adjacent to the first magnetic particle.

23. The method of claim 22 wherein said second magnetic particle moves at a velocity different than said first magnetic particle.

24. The method of claim 22 wherein said second magnetic particle has a different hydrodynamic volume or magnetic susceptibility different from the first magnetic particle.

25. The method of claim 22 wherein the second magnetic particle comprises a binding agent bound to a second target material.

26. The method of claim 22 wherein the second target material comprises a single stranded DNA fragment or an antigen.

27. The method of claim 22 wherein the second target material is selected from group consisting of: proteins, peptides, carbohydrates polysaccharides, glycoproteins, lipids, hormones, receptors, antigens, allergens, antibodies, substrates, metabolites, cofactors, inhibitors, drugs, pharmaceuticals, nutrients, toxins, poisons, explosives, pesticides, chemical warfare agents, biohazardous agents, vitamins, heterocyclic aromatic compounds, carcinogens, mutagens, narcotics, amphetamines, barbiturates, hallucinogens, and waste products.

28. The method of claim 1 comprising adding the magnetic particle to a sample suspected of containing a target material of interest.

29. The method of claim 28 comprising using a magnetic source to partition any magnetic material in the sample.

30. The method of claim 28 comprising collecting any magnetic material from the sample.

31. A method of analyzing a sample suspected of comprises a target material of interest, said method comprising:
preparing a magnetic transducer comprising a Fe/Au nanoparticle functionalized with a first binding agent wherein the Fe/Au nanoparticle exhibits a first magnet moment;
adding the magnetic transducer to the sample in an amount sufficient to bind to a target material in the sample and yield a bound transducer complex having the target material bonded thereto; and
determining the magnetic moment exhibited by the Fe/Au nanoparticle of the bound transducer complex.

32. The method of claim 31 wherein the magnetic transducer comprises a plurality of Fe/Au nanoparticles.

33. The method of claim 31, wherein the first binding agent is bound to a first Fe/Au and to a second Fe/Au particle.

34. The method of claim 31 wherein the binding agent in the transducer is flexible in the sample.

35. The method of claim 31 wherein the binding agent in the bound transducer complex is constrained.

36. The method of claim 31 wherein the binding agent comprises a single stranded DNA fragment.

37. The method of claim 31 wherein the target material comprises a single stranded DNA fragment.

38. The method of claim 31 comprising comparing the magnetic moment of the magnetic transducer to the magnetic moment of the bound transducer complex.

39. The method of claim 31 wherein said determining the magnetic moment comprises observing the mobility of the bound transducer complex in a magnetic field.

40. A method of analyzing a sample for a target material, said method comprising:
preparing a magnetic transducer comprising a magnetic susceptible nanoparticle having at least one binding agent attached thereto said binding agent selected to bind to the target material in the sample;
providing a labeled binding partner capable of binding to the binding agent; and
adding the magnetic transducer and the labeled binding partner to the sample.

41. The method of claim 40 wherein the binding partner is bound to the magnetic transducer to provide a first bound transducer complex prior to being added to the sample.

42. The method of claim 40 wherein the magnetic transducer and the labeled binding partner are added to the sample separately.

43. The method of claim 40 wherein the binding partner comprises a Au nanoparticle having at least one organic group bonded thereto.

44. The method of claim 40 wherein the target material displaces the binding partner from the first bound transducer complex to yield a second transducer complex.

45. The method of claim 40 wherein the target material is an antibody or an antigen.

46. The method of claim 40 wherein the binding partner is an antibody or an antigen.

47. The method of claim 40 wherein the binding group is an antibody or an antigen.

48. The method of claim 40 wherein the target material is selected from the group consisting of: proteins, peptides, carbohydrates polysaccharides, glycoproteins, lipids, hormones, receptors, antigens, allergens, antibodies, substrates, metabolites, cofactors, inhibitors, drugs, pharmaceuticals, nutrients, toxins, poisons, explosives, pesticides, chemical warfare agents, biohazardous agents, vitamins, heterocyclic aromatic compounds, carcinogens, mutagens, narcotics, amphetamines, barbiturates, hallucinogens, and waste products.

49. A device for analyzing a sample suspected of containing a target material, said device comprising:
a container configured to retain at least a portion of the sample, said container comprising at least one wall;
a magnet disposed adjacent to the at least one wall; and
an optical detector positioned next to the container and configured to detect the present of one or more species in the sample.

50. The device of claim 49 wherein the detector is positioned next to the at least one wall and adjacent to the magnet.

51. The device of claim 49 wherein the detector is spaced from the magnet.

52. The device of claim 49 wherein the optical detector using transmission electron microscopic techniques to analyze for the presence of one or more species in the sample.


SEQUENCE LISTING (3 sequences)

SEQ ID NO: 1 - DNA, 15 bp, Artificial Sequence
  misc-binding site using 5′ alkyl thiolate - (CH2)6SH
gtcagtccgtcagtc

SEQ ID NO: 2 - DNA, 15 bp, Artificial Sequence
  misc-binding site using a 3′alkyl thiolate of - (CH2)6SH
atcctcaactctccg

SEQ ID NO: 3 - DNA, 24 bp, Artificial Sequence
  Linker DNA substrate
agagttgagcatgactgacggact